(19) United States
(12) Patent Application Publication
(10) Pub. No.: US 2024/0076746 A1
(43) Pub. Date: Mar. 7, 2024

(54) PREDICTION OF AN OUTCOME OF A COLORECTAL CANCER SUBJECT

(71) Applicant: KONINKLIJKE PHILIPS N.V., EINDHOVEN (NL)

(72) Inventors: Ralf Dieter Hoffmann, Brueggen (DE); Monique Stoffels, Eindhoven (NL)

(21) Appl. No.: 18/271,793

(22) PCT Filed: Jan. 5, 2022
(86) PCT No.: PCT/EP2022/050105

§ 371 Date: Jul. 11, 2023

(30) Foreign Application Priority Data
Jan. 12, 2021 (EP) 21151054.0

(51) Int. Cl. C12Q 1/6886; C12Q 1/6851

(52) U.S. Cl. CPC C12Q 1/6886; C12Q 1/6851; C12Q 2600/118; C12Q 2600/158

(57) ABSTRACT

The invention relates to a method of predicting an outcome of a colorectal cancer subject, comprising determining or receiving the result of a determination of a first gene expression profile for each of one or more immune defense response genes, and/or of a second gene expression profile for each of one or more T-Cell receptor signaling genes, and/or of a third gene expression profile for each of one or more PDE4D7 correlated genes, said first, second, and third expression profile(s) being determined in a biological sample obtained from the subject, determining the prediction of outcome based on the first gene expression profile(s), or on the second gene expression profile(s), or on the third gene expression profile(s), or on the first, second, and third gene expression profile(s), and, optionally, providing the prediction to a medical caregiver or the subject.

### FIELD OF THE INVENTION

The invention relates to a method of predicting an outcome of a colorectal cancer subject, and to an apparatus for predicting an outcome of a colorectal cancer subject. Moreover, the invention relates to a diagnostic kit, to a use of the kit, to a use of the kit in a method of predicting an outcome of a colorectal cancer subject, to a use of first, second, and/or third gene expression profile(s) in a method of predicting an outcome of a colorectal cancer subject, and to a corresponding computer program product.

### BACKGROUND OF THE INVENTION

Cancer is a class of diseases in which a group of cells displays uncontrolled growth, invasion and sometimes metastasis. These three malignant properties of cancers differentiate them from benign tumours, which are self-limited and do not invade or metastasize. Colorectal cancer (CRC) is fairly common cancer, ranking 4^{th} (8.2%) in representing new cases, and even ranking 2^{nd} in number of estimated deaths for 2020 in the US. For 2020, it is estimated that almost 148,000 new cases of colorectal cancer will be diagnosed, and more than 53,000 will die from the disease. The median age of diagnosis is 67 with a 5-year relative survival of 64.6%. The earlier colorectal cancer is diagnosed, the higher the chance of surviving more than 5 years after diagnosis; the 5-year survival for localized colorectal cancer is 90.2%, while only 14.3% of patients with distant colorectal cancer survive for at least 5 years. About 38% of the patients present with localized disease. Men are affected more than women, and incidence increases with age (see National Cancer Institute, SEER Cancer Stat Facts: Colorectal Cancer, https://seer.cancer.gov/statfacts/html/colorect.html, accessed 09/09/2020). Risk factors for colorectal cancer include older age, having a family history of colorectal cancer in a first-degree relative, having a personal history of cancer of the colon, rectum, or ovary, having a personal history of high-risk adenomas, having inherited changes in certain genes that increase the risk of familial adenomatous polyposis (FAP) or Lynch syndrome, having a personal history of chronic ulcerative colitis or Crohn disease for +8 years, having >3 alcoholic drinks per day, smoking, being black, and being obese. In the past two decades, the rate of new cases is declining, although faster than the decline in the death rate.

Colorectal cancer starts in the colon or the rectum and can also be called colon cancer or rectal cancer, depending on where it started. Nevertheless, they are often grouped together because they have many features in common. Usually, CRC starts as a growth of the inner lining of the colon or rectum, called polyps. Whether these polyps change into cancer depends on the type of polyp. Adenomas sometimes change into cancer. Hyperplastic polyps and inflammatory polyps are more common, but generally not pre-cancerous. Sessile serrated polyps (SSP) and traditional serrated adenomas (TSA) are often treated as adenomas as they have a higher risk of colorectal cancer. In addition to the type of polyp, larger size of the polyp, multiple polyps, and dysplasia present in the polyp increase the risk of developing CRC.

Most CRCs are adenocarcinomas, which start in the cells that produce the mucus to lubricate the inside of the colon and rectum. Other, less common, types are carcinoid tumors (staring in hormone producing cells), Gastro-intestinal stromal tumors (GIST), lymphomas, and sarcomas.

Local treatments are more useful in early stage cancers. Depending on the type of CRC also systemic treatment may be given. For each type and stage different treatment types may be used, or combined at the same time, or used after one another. Some main treatment types are described at American Cancer Society, Colorectal Cancer, https://www.cancer.org/cancer/colon-rectal-cancer.html, accessed Sep. 9, 2022 and National Cancer Institute, PDQ® Adult Treatment Editorial Board, PDQ Colorectal Cancer Treatment, https://www.cancer.gov/types/colorectal/patient/colon-treatment-pdq, accessed 09/09/2020.

The main treatment for early stage colorectal cancers is surgery. The type of surgery depends on the grade of the cancer. For rectal cancer, radiation and chemotherapy are often given before or after surgery. Small metastases can be removed by embolization or ablation. This is also an option for patients whose cancer cannot be cured with surgery or who cannot have surgery. Radiation therapy is not common in colon cancer treatment but may be used in special cases, such as in (neo)adjuvant setting combined with surgery, or to treat metastases. For rectal cancer, radiation therapy is much more common and can be used before, after or during the surgery. It can also be used with or without chemotherapy in case surgery is not possible. Chemotherapy may be used at different times during treatment of colorectal cancer, in a (neo)adjuvant setting or to treat metastases. It can be given systemically, or regionally. Targeted therapies used in colorectal cancer can be targeted at: VEGF to prevent blood vessel formation, EGFR and BRAF to prevent cancer growth. Kinase inhibitors can also be used to slow cancer cell growth. Immune checkpoint inhibitors (PD-1 inhibitors, CTLA4-inhibitors) can be used for people whose colorectal cancer cells have tested positive to specific gene changes, such as microsatellite instability (MSI-H) or mismatch repair (MMR) genes. These drugs are often used when surgery is not possible, or for recurring or metastasized CRC.

Traditional classification of tumours has been refined by comprehensive molecular characterizations using large numbers of tumours. Colorectal cancers can be sub grouped by means of chromosomal instability (CIN), microsatellite instability (MSI) and hypermethylation. The cancer genome Atlas Network published a comprehensive molecular characterization of CRC in 2012 (see Cancer Genome Atlas Network, “Comprehensive molecular characterization of human colon and rectal cancer”, Nature, Vol. 487, No. 7407, pages 330-337, 2012). Many studies before have reported on genes and pathways important to initiation and progression of CRC, such as WNT, RAS-MAPK, PI3K, TGF-β, P53, and DNA mismatch repair pathways. The TCGA study concluded that non-hypermutated adenocarcinomas of the colon and rectum are not distinguishable at the genomic level. Activation of the WNT signalling pathway and inactivation of TGF-β signalling pathway, resulting in decreased activity of MYC, are nearly ubiquitous in CRC. Genomic aberrations frequently target PI3K and MAPK pathways, and less frequently receptor tyrosine kinases. Later, Liu Y. et al. provided a systematic effort to characterize shared molecular processes in gastrointestinal cancers (including CRC) (Liu Y. et al., “Comparative molecular analysis of gastrointestinal adenocarcinomas”, Cancer Cell, Vol. 33, No. 4, pages 721-735, 2018). Particularly in the lower GI tract, they found enriched activation of WNT signalling. Also, disruptions in TGF-β and SMAD signalling components were found, consistent with previous findings. Consideration of the molecular subtypes will be essential to study and treat CRC. For more granular details please refer to Liu Y. et al., 2018, ibid and Cancer Genome Atlas Network, 2018, ibid).

For example, EP 1777523A1, WO2007/082099A2, He et al. 2020 (Journal of Cancer, col. 11, no. 4, 1 Jan. 2020, pages 893-905), He et al. 2019 (Translational Cancer Research, vol. 8, no. 4, 1 Aug. 2019, pages 1351-1363), Sheelu et al. (Annals of Surgical Oncology, vol. 14, no. 12, 25 Sep. 2007, pages 3460-3471) and WO 2016/109546A2 describe marker genes for colorectal cancer.

The prediction of the therapy outcome is very complicated as many factors play a role in therapy effectiveness and disease recurrence. It is likely that important factors have not yet been identified, while the effect of others cannot be determined precisely. Multiple clinico-pathological measures are currently investigated and applied in a clinical setting to improve response prediction and therapy selection, providing some degree of improvement. Nevertheless, a strong need remains for better prediction of the treatment response, in order to increase the success rate of these therapies.

### SUMMARY OF THE INVENTION

It is an objective of the invention to provide a method of predicting an outcome of a colorectal cancer subject, and to an apparatus for predicting an outcome of a colorectal cancer subject, which allow to make better treatment decisions. It is a further aspect of the invention to provide a diagnostic kit, a use of the kit, a use of the kit in a method of predicting an outcome of a colorectal cancer subject, a use of first, second, and/or third gene expression profile(s) in a method of predicting an outcome of a colorectal cancer subject, and a corresponding computer program product.

In a first aspect of the present invention, a method of predicting an outcome of a colorectal cancer subject is presented, comprising:
determining or receiving the result of a determination of six or more gene expression levels selected from a first gene expression profile, a second gene expression profile and a third gene expression profile, wherein said six or more gene expression levels comprise at least one or more gene expression level selected from the third gene expression profile and at least one or more gene expression level selected from the first and/or the second gene expression profile, wherein
the first gene expression profile consist of the immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1;
the second gene expression profile consist of the T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70;
the third gene expression profile consists of the PDE4D7 correlated genes selected from the group consisting of: ABCC5, CUX2, KIAA1549, PDE4D, RAP1GAP2, SLC39A11, TDRD1, and VWA2;
said first, second and third gene expression profiles being determined in a biological sample obtained from the subject; and
determining the prediction of the outcome based on six or more gene expression levels, and
optionally, providing the prediction to a medical caregiver or the subject. In an embodiment the six or more gene expression levels comprise one or more, for example, 1, 2, 3, 4, 5, 6, 7, 8, 9, 10, 11, 12, 13 or all, immune defense response genes, and one or more, for example, 1, 2, 3, 4, 5, 6, 7, 8, 9, 10, 11, 12, 13 or all, T-Cell receptor signaling genes, and one or more, for example, 1, 2, 3, 4, 5, 6, 7, 8, 9, 10, 11, 12, 13 or all, PDE4D7 correlated genes.

Alternatively, the invention relates to a method of predicting an outcome of a colorectal cancer subject is presented, comprising:
determining or receiving the result of a determination of a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, said first gene expression profile(s) being determined in a biological sample obtained from the subject, and/or
determining or receiving the result of a determination of a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, said second gene expression profile(s) being determined in a biological sample obtained from the subject, and/or
determining or receiving the result of a determination of a third gene expression profile for each of one or more, for example, 1, 2, 3, 4, 5, 6, 7 or all, PDE4D7 correlated genes selected from the group consisting of: ABCC5, CUX2, KIAA1549, PDE4D, RAP1GAP2, SLC39A11, TDRD1, and VWA2, said third gene expression profile(s) being determined in a biological sample obtained from the subject,
determining the prediction of the outcome based on the first gene expression profile(s), or on the second gene expression profile(s), or on the third gene expression profile(s), or on the first, second, and third gene expression profile(s), and
optionally, providing the prediction to a medical caregiver or the subject.

In recent years, the importance of the immune system in cancer inhibition as well as in cancer initiation, promotion and metastasis has become very evident (see Mantovani A. et al., “Cancer-related inflammation”, Nature, Vol. 454, No. 7203, pages 436-444, 2008, and Giraldo N. A. et al., “The clinical role of the TME in solid cancer”, Br J Cancer, Vol. 120, No. 1, pages 45-53, 2019). The immune cells and the molecules they secrete form a crucial part of the tumour microenvironment and most immune cells can infiltrate the tumour tissue. The immune system and the tumour affect and shape one another. Thus, anti-tumour immunity can prevent tumour formation while an inflammatory tumour environment may promote cancer initiation and proliferation. At the same time, tumour cells that may have originated in an immune system-independent manner will shape the immune microenvironment by recruiting immune cells and can have a pro-inflammatory effect while also suppressing anti-cancer immunity.

Some of the immune cells in the tumour microenvironment will have either a general tumour-promoting or a general tumour-inhibiting effect, while other immune cells exhibit plasticity and show both tumour-promoting and tumour-inhibiting potential. Thus, the overall immune microenvironment of the tumour is a mixture of the various immune cells present, the cytokines they produce and their interactions with tumour cells and with other cells in the tumour microenvironment (see Giraldo N. A. et al., 2019, ibid).

The principles described above with regard to the role of the immune system in cancer in general also apply to prostate cancer. Chronic inflammation has been linked to the formation of benign as well as malignant prostate tissue (see Hall W. A. et al., 2016, ibid) and most prostate cancer tissue samples show immune cell infiltrates. The presence of specific immune cells with a pro-tumour effect has been correlated with worse prognosis, while tumours in which natural killer cells were more activated showed better response to therapy and longer recurrence-free periods (see Shiao S. L. et al., “Regulation of prostate cancer progression by tumor microenvironment”, Cancer Lett, Vol. 380, No. 1, pages 340-348, 2016).

While a therapy will be influenced by the immune components of the tumour microenvironment, RT itself extensively affects the make-up of these components (see Barker H. E. et al., “The tumor microenvironment after radiotherapy: Mechanisms of resistance or recurrence”, Nat Rev Cancer, Vol. 15, No. 7, pages 409-425, 2015). Because suppressive cell types are comparably radiation-insensitive, their relative numbers will increase. Counteractively, the inflicted radiation damage activates cell survival pathways and stimulates the immune system, triggering inflammatory responses and immune cell recruitment. Whether the net effect will be tumour-promoting or tumour-suppressing is as yet uncertain, but its potential for enhancement of cancer immunotherapies is being investigated.

The present invention is based on the idea that, since the status of the immune system and of the immune microenvironment have an impact on therapy effectiveness, the ability to identify markers predictive for this effect might help to be better able to predict the outcome of a colorectal cancer subject.

Herein, the term “outcome” relates to a specific result or effect that can be measured. Examples of an outcome of a colorectal cancer subject include an outcome of the colorectal cancer, a pathology outcome, an outcome of a therapy for treating the colorectal cancer, such as a surgery outcome, a radiation therapy outcome, a chemotherapy outcome, and an immunotherapy outcome, as well as other outcomes, such as a biomarker related outcome (e.g., CEA (Carcinoembryonic Antigen)), a genomic profile related outcome, an imaging related outcome (e.g., a change in morphology or texture of the tumor), a biology related outcome (e.g., inflammation or immune response), a surrogate marker related outcome, a tumor size outcome, a treatment side effect outcome, a treatment toxicity outcome, a disease pain outcome, a quality of life outcome, a cancer specific survival, and an overall survival.

### Immune Response Defense Genes

The integrity and stability of genomics DNA is permanently under stress induced by various cell internal and external factors like exposure to radiation, viral or bacterial infections, but also oxidation and replication stress (see Gasser S. et al., “Sensing of dangerous DNA”, Mechanisms of Aging and Development, Vol. 165, pages 33-46, 2017). In order to maintain DNA structure and stability, a cell must be able to recognize all types of DNA damages like single or double strand breaks etc. induced by various factors. This process involves the participation of a multitude of specific proteins depending on the kind of damage as part of DNA recognition pathways.

Recent evidence suggests that mis-localized DNA (e.g., DNA unnaturally appearing in the cytosolic fraction of the cell in contrast to the nucleus) and damaged DNA (e.g., through mutations occurring in cancer development) is used by the immune system to identify infected or otherwise diseased cells while genomic and mitochondrial DNA present in healthy cells is ignored by DNA recognition pathways. In diseased cells, cytosolic DNA sensor proteins have been demonstrated to be involved in the detection of DNA occurring unnaturally in the cytosol of the cell. Detection of such DNA by different nucleic acid sensors translates into similar responses leading to nuclear factor kappa-B (NF-kB) and interferon type I (IFN type I) signalling followed by the activation of innate immune system components. While the recognition of viral DNA is known to induce an IFN type I response, evidence that sensing of DNA damage can initiate immune responses has only recently been accumulating.

TLR9 (Toll-like receptor 9) located in the endosomes was one of the first DNA sensors molecules identified to be involved in the immune recognition of DNA by signalling downstream via the adaptor protein myeloid differentiation primary-response protein88 (MYD88). This interaction in turn activates mitogen-activated protein kinases (MAPKs) and NF-kB. TLR9 also induces the generation of type I interferons through the activation of IRF7 via IkB Kinase alpha (IKKalpha) in plasmacytoid dendritic cells (pDCs). Various other DNA immune receptors including IF116 (IFN-gamma-inducible protein 16), cGAS (cyclic DMP-AMP synthase, DDX41 (DEAD-box helicase 41), as well as ZBP1 (Z-DNA-binding protein 1) interact with STING (stimulator of IFN genes), which activates the IKK complex and IRF3 through TBK1 (TANK binding kinase 1). ZBP1 also activates NF-kB via recruitment of RIP1 and RIP3 (receptor-interacting protein 1 and 3, respectively). While the helicase DHX36 (DEAH-box helicase 36) interacts in a complex with TRID to induce NF-kB and IRF-3/7 the DHX9 helicase stimulates MYD88-dependent signalling in plasmacytoid dendritic cells. The DNA sensor LRRFIP1 (leucine-rich repeat flightless-interacting protein) complexes with beta-catenin to activate the transcription of IRF3 whereas AIM2 (absent in melanoma 2) recruits the adaptor protein ASC (apoptosis speck-like protein) to induce a caspase-1-activating inflammasome complex leading to the secretion of interleukin-Ibeta (IL-1beta) and IL-18 (see FIG. 1 of Gasser S. et al., 2017, ibid, which provides a schematic overview of DNA damage and DNA sensor pathways leading to the production of inflammatory cytokines and the expression of ligands for activating innate immune receptors. Members of the non-homologous end joining pathway (orange), homologous recombination (red), inflammasome (dark green), NF-kB and interferon responses (light green) are shown).

The factors and mechanisms responsible for activating the DNA sensor pathways in cancer are currently not well elucidated. It will be important to identify the intratumoral DNA species, sensors and pathways implicated in the expression of IFNs in different cancer types at all stages of the disease. In addition to therapeutic targets in cancer, such factors may also have prognostic and predictive value. Novel DNA sensor pathway agonists and antagonists are currently being developed and tested in preclinical trials. Such compounds will be useful in characterizing the role of DNA sensor pathways in the pathogenesis of cancer, autoimmunity and potentially other diseases.

### T-Cell Receptor Signaling Genes

An immune response against pathogens can be elicited at different levels: there are physical barriers, such as the skin, to keep invaders out. If breached, innate immunity comes into play; a first and fast non-specific response. If this is not sufficient, the adaptive immune response is elicited. This is much more specific and needs time to develop when encountering a pathogen for the first time. Lymphocytes are activated by interacting with activated antigen presenting cells from the innate immune system, and are also responsible for maintenance of memory for faster responses upon next encounters with the same pathogen.

As lymphocytes are highly specific and effective when activated, they are subject to negative selection for their ability to recognize self, a process known as central tolerance. As not all self-antigens are expressed at selection sites, peripheral tolerance mechanisms evolved as well, such as ligation of the TCR in absence of co-stimulation, expression of inhibitory co-receptors, and suppression by Tregs. A disturbed balance between activation and suppression may lead to autoimmune disorders, or immune deficiencies and cancer, respectively.

T-cell activation can have different functional consequences, depending on the location the type of T-cell involved. CD8+ T-cells differentiate into cytotoxic effector cells, whereas CD4+ T-cells can differentiate into Th1 (IFNγ secretion and promotion of cell mediated immunity) or Th2 (IL4/5/13 secretion and promotion of B cell and humoral immunity). Differentiation towards other, more recently identified T-cell subsets is also possible, for example the Tregs, which have a suppressive effect on immune activation (see Mosenden R. and Tasken K., “Cyclic AMP-mediated immune regulation—Overview of mechanisms of action in T-cells”, Cell Signal, Vol. 23, No. 6, pages 1009-1016, 2011, in particular, FIG. 4, which T-cell activation and its modulation by PKA, and Tasken K. and Ruppelt A., “Negative regulation of T-cell receptor activation by the cAMP-PKA-Csk signaling pathway in T-cell lipid rafts”, Front Biosci, Vol. 11, pages 2929-2939, 2006).

Both PKA and PDE4 regulated signaling intersect with TCR induced T-cell activation to fine-tune its regulation, with opposing effects (see Abrahamsen H. et al., “TCR- and CD28-mediated recruitment of phosphodiesterase 4 to lipid rafts potentiates TCR signaling”, J Immunol, Vol. 173, pages 4847-4848, 2004, in particular, FIG. 6, which shows opposing effects of PKA and PDE4 on TCR activation). The molecule that connects these effectors is cyclic AMP (cAMP), an intracellular second messenger of extracellular ligand action. In T-cells, it mediates effects of prostaglandins, adenosine, histamine, beta-adrenergic agonists, neuropeptide hormones and beta-endorphin. Binding of these extracellular molecules to GPCRs leads to their conformational change, release of stimulatory subunits and subsequent activation of adenylate cyclases (AC), which hydrolyze ATP to cAMP (see FIG. 6 of Abrahamsen H. et al., 2004, ibid). Although not the only one, PKA is the principal effector of cAMP signaling (see Mosenden R. and Tasken K., 2011, ibid, and Tasken K. and Ruppelt A., 2006, ibid). At a functional level, increased levels of cAMP lead to reduced IFNγ and IL-2 production in T-cells (see Abrahamsen H. et al., 2004, ibid). Aside from interfering with TCR activation, PKA has many more effector (see FIG. 15 of Torheim E. A., “Immunity Leashed—Mechanisms of Regulation in the Human Immune System”, Thesis for the degree of Philosophiae Doctor (PhD), The Biotechnology Centre of Ola, University of Oslo, Norway, 2009).

In naïve T-cells, hyperphosphorylated PAG targets Csk to lipid rafts. Via the Ezrin-EBP50-PAG scaffold complex PKA is targeted to Csk. Through specific phosphorylation by PKA, Csk can negatively regulate Lek and Fyn to dampen their activity and downregulate T-cell activation (see FIG. 6 of Abrahamsen H. et al., 2004, ibid). Upon TCR activation, PAG is dephosphorylated and Csk is released from the rafts. Dissociation of Csk is needed for T-cell activation to proceed. Within the same time course, a Csk-G3BP complex is formed and seems to sequester Csk outside lipid rafts (see Mosenden R. and Tasken K., 2011, ibid, and Tasken K. and Ruppelt A., 2006, ibid).

In contrast, combined TCR and CD28 stimulation mediates recruitment of the cyclic nucleotide phosphodiesterase PDE4 to lipid rafts, which enhances cAMP degradation (see FIG. 6 of Abrahamsen H. et al., 2004, ibid). As such, TCR induced production of cAMP is countered, and the T-cell immune response potentiated. Upon TCR stimulation alone, PDE4 recruitment may be too low to fully reduce the cAMP levels and therefore maximal T-cell activation cannot occur (see Abrahamsen H. et al., 2004, ibid).

Thus, by active suppression of proximal TCR signaling, signaling via cAMP-PKA-Csk is thought to set the threshold for T-cell activation. Recruitment of PDEs can counter this suppression. Tissue or cell-type specific regulation is accomplished through expression of multiple isoforms of AC, PKA, and PDEs. As mentioned above, the balance between activation and suppression needs to be tightly regulated to prevent development of autoimmune disorders, immune deficiencies and cancer.

### PDE4D7 Correlated Genes

Phosphodiesterases (PDEs) provide the sole means for the degradation of the second messenger 3′-5′-cyclic AMP. As such they are poised to provide a key regulatory role. Thus, aberrant changes in their expression, activity and intracellular location may all contribute to the underlying molecular pathology of particular disease states. Indeed, it has recently been shown that mutations in PDE genes are enriched in prostate cancer patients leading to elevated cAMP signalling and a potential predisposition to prostate cancer. However, varied expression profiles in different cell types coupled with complex arrays of isoform variants within each PDE family makes understanding the links between aberrant changes in PDE expression and functionality during disease progression challenging. Several studies have endeavored to describe the complement of PDEs in prostate, all of which identified significant levels of PDE4 expression alongside other PDEs, leading to the development of a PDE4D7 biomarker (see Alves de Inda M. et al., “Validation of Cyclic Adenosine Monophosphate Phosphodiesterase-4D7 for its Independent Contribution to Risk Stratification in a Prostate Cancer Patient Cohort with Longitudinal Biological Outcomes”, Eur Urol Focus, Vol. 4, No. 3, pages 376-384, 2018). Since the PDE4D7 biomarker has been proven to be a good predictor, we assumed that the ability to identify markers that are highly correlated with the PDE47 biomarker might also be helpful in prognosticating the outcome of certain cancer subjects.

### Selection of the Genes

The lists of genes have originally been selected by us for prognostication in prostate cancer subjects. In this document, it is shown that they are also of prognostic value with respect to an outcome of a colorectal cancer subject.

The identified immune defense response genes ZBP1, and AIM2, APOBEC3A, CIAO1, DDX58, DHX9, IFI16, IFIH1, IFIT1, IFIT3, LRRFIP1, MYD88, OAS1, TLR8, and ZBP1, respectively, were identified as follows: A group of 538 prostate cancer patients were treated with RP and the prostate cancer tissue was stored together with clinical (e.g., pathological Gleason grade group (pGGG), pathology state (pT stage)) as well as relevant outcome parameters (e.g., biochemical recurrence (BCR), metastatic recurrence, prostate cancer specific death (PCa death), salvage radiation treatment (SRT), salvage androgen deprivation treatment (SADT), chemotherapy (CTX)). For each of these patients, a PDE4D7 score was calculated and categorized into four PDE4D7 score classes (see Alves de Inda M. et al., 2018, ibid). PDE4D7 score class 1 represents patient samples with lowest expression levels of PDE4D7, whereas PDE4D7 score class 4 represents patient samples with highest levels of PDE4D7 expression. RNASeq expression data (TPM—Transcripts Per Million) of the 538 prostate cancer subjects was then investigated for differential gene expression between the PDE4D7 score classes 1 and 4. In particular, it was determined for around 20,000 protein coding transcripts whether the mean expression level of the PDE4D7 score class 1 patients was more than twice as high as the mean expression level of the PDE4D7 score class 4 patients. This analysis resulted in 637 genes with a ratio PDE4D7 score class 1/PDE4D7 score class 4 of >2 with a minimum mean expression of 1 TPM in each of the four PDE4D7 score classes. These 637 genes were then further subjected to molecular pathway analysis (www.david.ncifcrf.gov), which resulted in a range of enriched annotation clusters. The annotation cluster #2 demonstrated enrichment (enrichment score: 10.8) in 30 genes with a function in defense response to viruses, negative regulation of viral genome replication as well as type I interferon signaling. A further heat map analysis confirmed that these immune defense response genes were generally higher expressed in samples from patients in PDE4D7 score class 1 than from patients in PDE4D7 score class 4. The class of genes with a function in defense response to viruses, negative regulation of viral genome replication as well as type I interferon signaling was further enriched to 61 genes by literature search to identify additional genes with the same molecular function. A further selection from the 61 genes was made based on the combinatorial power to separate patients who died from prostate cancer vs. those who did not, resulting of a preferred set of 14 genes. It was found that the number of events (metastases, prostate cancer specific death) was enriched in sub-cohorts with a low expression of these genes compared to the total patient cohort (#538) and a sub-cohort of 151 patients undergoing salvage RT (SRT) after post-surgical disease recurrence.

The identified T-Cell receptor signaling genes CD2, CD247, CD28, CD3E, CD3G, CD4, CSK, EZR, FYN, LAT, LCK, PAG1, PDE4D, PRKACA, PRKACB, PTPRC, and ZAP70 were identified as follows: A group of 538 prostate cancer patients were treated with RP and the prostate cancer tissue was stored together with clinical (e.g., pathological Gleason grade group (pGGG), pathology state (pT stage)) as well as relevant outcome parameters (e.g., biochemical recurrence (BCR), metastatic recurrence, prostate cancer specific death (PCa death), salvage radiation treatment (SRT), salvage androgen deprivation treatment (SADT), chemotherapy (CTX)). For each of these patients, a PDE4D7 score was calculated and categorized into four PDE4D7 score classes (see Alves de Inda M. et al., 2018, ibid). PDE4D7 score class 1 represents patient samples with lowest expression levels of PDE4D7, whereas PDE4D7 score class 4 represents patient samples with highest levels of PDE4D7 expression. RNASeq expression data (TPM—Transcripts Per Million) of the 538 prostate cancer subjects was then investigated for differential gene expression between the PDE4D7 score classes 1 and 4. In particular, it was determined for around 20,000 protein coding transcripts whether the mean expression level of the PDE4D7 score class 1 patients was more than twice as high as the mean expression level of the PDE4D7 score class 4 patients. This analysis resulted in 637 genes with a ratio PDE4D7 score class 1/PDE4D7 score class 4 of >2 with a minimum mean expression of 1 TPM in each of the four PDE4D7 score classes. These 637 genes were then further subjected to molecular pathway analysis (www.david.ncifcrf.gov), which resulted in a range of enriched annotation clusters. The annotation cluster #6 demonstrated enrichment (enrichment score: 5.9) in 17 genes with a function in primary immune deficiency and activation of T-Cell receptor signaling. A further heat map analysis confirmed that these T-Cell receptor signaling genes were generally higher expressed in samples from patients in PDE4D7 score class 1 than from patients in PDE4D7 score class 4.

The identified PDE4D7 correlated genes ABCC5, CUX2, KIAA1549, PDE4D, RAP1GAP2, SLC39A11, TDRD1, and VWA2 were identified as follows: We have identified in RNAseq data generated on 571 prostate cancer patients on close to 60,000 transcripts a range of genes that are correlated to the expression of the known biomarker PDE4D7 in this data. The correlation between the expression of any of these genes and PDE4D7 across the 571 samples was done by Pearson correlation and is expressed as a value between 0 to 1 in case of positive correlation or a value between −1 to 0 in case of negative correlation. As input data for the calculation of the correlation coefficient we used the PDE4D7 score (see Alves de Inda M. et al., 2018, ibid) and the RNAseq determined TPM gene expression value per gene of interest (see below).

The maximum negative correlation coefficient identified between the expression of any of the approximately 60,000 transcripts and the expression of PDE4D7 was −0.38 while the maximum positive correlation coefficient identified between the expression of any of the approximately 60,000 transcripts and the expression of PDE4D7 was +0.56. We selected genes in the range of correlation −0.31 to −0.38 as well as +0.41 to +0.56. We identified in total 77 transcripts matching these characteristics. From those 77 transcripts we selected the eight PDE4D7 correlated genes ABCC5, CUX2, KIAA1549, PDE4D, RAP1GAP2, SLC39A11, TDRD1, and VWA2 by testing Cox regression combination models iteratively in a sub-cohort of 186 patients who were undergoing salvage radiation treatment (SRT) due to post-surgical biochemical relapse. The clinical endpoint tested was prostate cancer specific death after start of SRT. The boundary condition for the selection of the eight genes was given by the restriction that the p-values in the multivariate Cox-regression were <0.1 for all genes retained in the model.

The term “ABCC5” refers to the human ATP binding cassette subfamily C member 5 gene (Ensembl: ENSG00000114770), for example, to the sequence as defined in NCBI Reference Sequence NM_001023587.2 or in NCBI Reference Sequence NM_005688.3, specifically, to the nucleotide sequence as set forth in SEQ ID NO:1 or in SEQ ID NO:2, which correspond to the sequences of the above indicated NCBI Reference Sequences of the ABCC5 transcript, and also relates to the corresponding amino acid sequence for example as set forth in SEQ ID NO:3 or in SEQ ID NO:4, which correspond to the protein sequences defined in NCBI Protein Accession Reference Sequence NP_001018881.1 and in NCBI Protein Accession Reference Sequence NP_005679 encoding the ABCC5 polypeptide.

The term “ABCC5” also comprises nucleotide sequences showing a high degree of homology to ABCC5, e.g., nucleic acid sequences being at least 75%, 80%, 85%, 90%, 91%, 92%, 93%, 94%, 95%, 96%, 97%, 98% or 99% identical to the sequence as set forth in SEQ ID NO:1 or in SEQ ID NO:2 or amino acid sequences being at least 75%, 80%, 85%, 90%, 91%, 92%, 93%, 94%, 95%, 96%, 97%, 98% or 99% identical to the sequence as set forth in SEQ ID NO:3 or in SEQ ID NO:4 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:3 or in SEQ ID NO:4 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:1 or in SEQ ID NO:2.

The term “AIM2” refers to the Absent in Melanoma 2 gene (Ensembl: ENSG00000163568), for example, to the sequence as defined in NCBI Reference Sequence NM_004833, specifically, to the nucleotide sequence as set forth in SEQ ID NO:5, which corresponds to the sequence of the above indicated NCBI Reference Sequence of the AIM2 transcript, and also relates to the corresponding amino acid sequence for example as set forth in SEQ ID NO:6, which corresponds to the protein sequence defined in NCBI Protein Accession Reference Sequence NP_004824 encoding the AIM2 polypeptide.

The term “AIM2” also comprises nucleotide sequences showing a high degree of homology to AIM2, e.g., nucleic acid sequences being at least 75%, 80%, 85%, 90%, 91%, 92%, 93%, 94%, 95%, 96%, 97%, 98% or 99% identical to the sequence as set forth in SEQ ID NO:5 or amino acid sequences being at least 75%, 80%, 85%, 90%, 91%, 92%, 93%, 94%, 95%, 96%, 97%, 98% or 99% identical to the sequence as set forth in SEQ ID NO:6 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:6 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:5.

The term “APOBEC3A” refers to the Apolipoprotein B mRNA Editing Enzyme Catalytic Subunit 3A gene (Ensembl: ENSG00000128383), for example, to the sequence as defined in NCBI Reference Sequence NM_145699, specifically, to the nucleotide sequence as set forth in SEQ ID NO:7, which corresponds to the sequence of the above indicated NCBI Reference Sequence of the APOBEC3A transcript, and also relates to the corresponding amino acid sequence for example as set forth in SEQ ID NO:8, which corresponds to the protein sequence defined in NCBI Protein Accession Reference Sequence NP_663745 encoding the APOBEC3A polypeptide.

The term “APOBEC3A” also comprises nucleotide sequences showing a high degree of homology to APOBEC3A, e.g., nucleic acid sequences being at least 75%, 80%, 85%, 90%, 91%, 92%, 93%, 94%, 95%, 96%, 97%, 98% or 99% identical to the sequence as set forth in SEQ ID NO:7 or amino acid sequences being at least 75%, 80%, 85%, 90%, 91%, 92%, 93%, 94%, 95%, 96%, 97%, 98% or 99% identical to the sequence as set forth in SEQ ID NO:8 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:8 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:7.

The term “CD2” refers to the Cluster Of Differentiation 2 gene (Ensembl: ENSG00000116824), for example, to the sequence as defined in NCBI Reference Sequence NM_001767, specifically, to the nucleotide sequence as set forth in SEQ ID NO:9, which corresponds to the sequence of the above indicated NCBI Reference Sequence of the CD2 transcript, and also relates to the corresponding amino acid sequence for example as set forth in SEQ ID NO:10, which corresponds to the protein sequence defined in NCBI Protein Accession Reference Sequence NP_001758 encoding the CD2 polypeptide.

The term “CD2” also comprises nucleotide sequences showing a high degree of homology to CD2, e.g., nucleic acid sequences being at least 75%, 80%, 85%, 90%, 91%, 92%, 93%, 94%, 95%, 96%, 97%, 98% or 99% identical to the sequence as set forth in SEQ ID NO:9 or amino acid sequences being at least 75%, 80%, 85%, 90%, 91%, 92%, 93%, 94%, 95%, 96%, 97%, 98% or 99% identical to the sequence as set forth in SEQ ID NO:10 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:10 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:9.

The term “CD247” refers to the Cluster Of Differentiation 247 gene (Ensembl: ENSG00000198821), for example, to the sequence as defined in NCBI Reference Sequence NM_000734 or in NCBI Reference Sequence NM_198053, specifically, to the nucleotide sequence as set forth in SEQ ID NO:11 or in SEQ ID NO:12, which correspond to the sequences of the above indicated NCBI Reference Sequences of the CD247 transcript, and also relates to the corresponding amino acid sequence for example as set forth in SEQ ID NO:13 or in SEQ ID NO:14, which correspond to the protein sequences defined in NCBI Protein Accession Reference Sequence NP_000725 and in NCBI Protein Accession Reference Sequence NP_932170 encoding the CD247 polypeptide.

The term “CD247” also comprises nucleotide sequences showing a high degree of homology to CD247, e.g., nucleic acid sequences being at least 75%, 80%, 85%, 90%, 91%, 92%, 93%, 94%, 95%, 96%, 97%, 98% or 99% identical to the sequence as set forth in SEQ ID NO:11 or in SEQ ID NO:12 or amino acid sequences being at least 75%, 80%, 85%, 90%, 91%, 92%, 93%, 94%, 95%, 96%, 97%, 98% or 99% identical to the sequence as set forth in SEQ ID NO:13 or in SEQ ID NO:14 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:13 or in SEQ ID NO:14 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:11 or in SEQ ID NO:12.

The term “CD28” refers to the Cluster Of Differentiation 28 gene (Ensembl: ENSG00000178562), for example, to the sequence as defined in NCBI Reference Sequence NM_006139 or in NCBI Reference Sequence NM_001243078, specifically, to the nucleotide sequence as set forth in SEQ ID NO:15 or in SEQ ID NO:16, which correspond to the sequences of the above indicated NCBI Reference Sequences of the CD28 transcript, and also relates to the corresponding amino acid sequence for example as set forth in SEQ ID NO:17 or in SEQ ID NO:18, which correspond to the protein sequences defined in NCBI Protein Accession Reference Sequence NP_006130 and in NCBI Protein Accession Reference Sequence NP_001230007 encoding the CD28 polypeptide.

The term “CD28” also comprises nucleotide sequences showing a high degree of homology to CD28, e.g., nucleic acid sequences being at least 75%, 80%, 85%, 90%, 91%, 92%, 93%, 94%, 95%, 96%, 97%, 98% or 99% identical to the sequence as set forth in SEQ ID NO:15 or in SEQ ID NO:16 or amino acid sequences being at least 75%, 80%, 85%, 90%, 91%, 92%, 93%, 94%, 95%, 96%, 97%, 98% or 99% identical to the sequence as set forth in SEQ ID NO:17 or in SEQ ID NO:18 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:17 or in SEQ ID NO:18 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:15 or in SEQ ID NO:16.

The term “CD3E” refers to the Cluster Of Differentiation 3E gene (Ensembl: ENSG00000198851), for example, to the sequence as defined in NCBI Reference Sequence NM_000733, specifically, to the nucleotide sequence as set forth in SEQ ID NO:19, which corresponds to the sequence of the above indicated NCBI Reference Sequence of the CD3E transcript, and also relates to the corresponding amino acid sequence for example as set forth in SEQ ID NO:20, which corresponds to the protein sequence defined in NCBI Protein Accession Reference Sequence NP_000724 encoding the CD3E polypeptide.

The term “CD3E” also comprises nucleotide sequences showing a high degree of homology to CD3E, e.g., nucleic acid sequences being at least 75%, 80%, 85%, 90%, 91%, 92%, 93%, 94%, 95%, 96%, 97%, 98% or 99% identical to the sequence as set forth in SEQ ID NO:19 or amino acid sequences being at least 75%, 80%, 85%, 90%, 91%, 92%, 93%, 94%, 95%, 96%, 97%, 98% or 99% identical to the sequence as set forth in SEQ ID NO:20 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:20 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:19.

The term “CD3G” refers to the Cluster Of Differentiation 3G gene (Ensembl: ENSG00000160654), for example, to the sequence as defined in NCBI Reference Sequence NM_000073, specifically, to the nucleotide sequence as set forth in SEQ ID NO:21, which corresponds to the sequence of the above indicated NCBI Reference Sequence of the CD3G transcript, and also relates to the corresponding amino acid sequence for example as set forth in SEQ ID NO:22, which corresponds to the protein sequence defined in NCBI Protein Accession Reference Sequence NP_000064 encoding the CD3G polypeptide.

The term “CD3G” also comprises nucleotide sequences showing a high degree of homology to CD3G, e.g., nucleic acid sequences being at least 75%, 80%, 85%, 90%, 91%, 92%, 93%, 94%, 95%, 96%, 97%, 98% or 99% identical to the sequence as set forth in SEQ ID NO:21 or amino acid sequences being at least 75%, 80%, 85%, 90%, 91%, 92%, 93%, 94%, 95%, 96%, 97%, 98% or 99% identical to the sequence as set forth in SEQ ID NO:22 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:22 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:21.

The term “CD4” refers to the Cluster Of Differentiation 4 gene (Ensembl: ENSG00000010610), for example, to the sequence as defined in NCBI Reference Sequence NM_000616, specifically, to the nucleotide sequence as set forth in SEQ ID NO:23, which corresponds to the sequence of the above indicated NCBI Reference Sequence of the CD4 transcript, and also relates to the corresponding amino acid sequence for example as set forth in SEQ ID NO:24, which corresponds to the protein sequence defined in NCBI Protein Accession Reference Sequence NP_000607 encoding the CD4 polypeptide.

The term “CD4” also comprises nucleotide sequences showing a high degree of homology to CD4, e.g., nucleic acid sequences being at least 75%, 80%, 85%, 90%, 91%, 92%, 93%, 94%, 95%, 96%, 97%, 98% or 99% identical to the sequence as set forth in SEQ ID NO:23 or amino acid sequences being at least 75%, 80%, 85%, 90%, 91%, 92%, 93%, 94%, 95%, 96%, 97%, 98% or 99% identical to the sequence as set forth in SEQ ID NO:24 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:24 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:23.

The term “CIAO1” refers to the Cytosolic Iron-Sulfur Assembly Component 1 gene (Ensembl: ENSG00000144021), for example, to the sequence as defined in NCBI Reference Sequence NM_004804, specifically, to the nucleotide sequence as set forth in SEQ ID NO:25, which corresponds to the sequence of the above indicated NCBI Reference Sequence of the CIAO1 transcript, and also relates to the corresponding amino acid sequence for example as set forth in SEQ ID NO:26, which corresponds to the protein sequence defined in NCBI Protein Accession Reference Sequence NP_663745 encoding the CIAO1 polypeptide.

The term “CIAO1” also comprises nucleotide sequences showing a high degree of homology to CIAO1, e.g., nucleic acid sequences being at least 75%, 80%, 85%, 90%, 91%, 92%, 93%, 94%, 95%, 96%, 97%, 98% or 99% identical to the sequence as set forth in SEQ ID NO:25 or amino acid sequences being at least 75%, 80%, 85%, 90%, 91%, 92%, 93%, 94%, 95%, 96%, 97%, 98% or 99% identical to the sequence as set forth in SEQ ID NO:26 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:26 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:25.

The term “CSK” refers to the C-Terminal Src Kinase gene (Ensembl: ENSG00000103653), for example, to the sequence as defined in NCBI Reference Sequence NM_004383, specifically, to the nucleotide sequence as set forth in SEQ ID NO:27, which corresponds to the sequence of the above indicated NCBI Reference Sequence of the CSK transcript, and also relates to the corresponding amino acid sequence for example as set forth in SEQ ID NO:28, which corresponds to the protein sequence defined in NCBI Protein Accession Reference Sequence NP_004374 encoding the CSK polypeptide.

The term “CSK” also comprises nucleotide sequences showing a high degree of homology to CSK, e.g., nucleic acid sequences being at least 75%, 80%, 85%, 90%, 91%, 92%, 93%, 94%, 95%, 96%, 97%, 98% or 99% identical to the sequence as set forth in SEQ ID NO:27 or amino acid sequences being at least 75%, 80%, 85%, 90%, 91%, 92%, 93%, 94%, 95%, 96%, 97%, 98% or 99% identical to the sequence as set forth in SEQ ID NO:28 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:28 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:27.

The term “CUX2” refers to the human Cut Like Homeobox 2 gene (Ensembl: ENSG00000111249), for example, to the sequence as defined in NCBI Reference Sequence NM_015267.3, specifically, to the nucleotide sequence as set forth in SEQ ID NO:29, which corresponds to the sequence of the above indicated NCBI Reference Sequence of the CUX2 transcript, and also relates to the corresponding amino acid sequence for example as set forth in SEQ ID NO:30, which corresponds to the protein sequence defined in NCBI Protein Accession Reference Sequence NP_056082.2 encoding the CUX2 polypeptide.

The term “CUX2” also comprises nucleotide sequences showing a high degree of homology to CUX2, e.g., nucleic acid sequences being at least 75%, 80%, 85%, 90%, 91%, 92%, 93%, 94%, 95%, 96%, 97%, 98% or 99% identical to the sequence as set forth in SEQ ID NO:29 or amino acid sequences being at least 75%, 80%, 85%, 90%, 91%, 92%, 93%, 94%, 95%, 96%, 97%, 98% or 99% identical to the sequence as set forth in SEQ ID NO:30 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:30 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:29.

The term “DDX58” refers to the DExD/H-box Helicase 58 gene (Ensembl: ENSG00000107201), for example, to the sequence as defined in NCBI Reference Sequence NM_014314, specifically, to the nucleotide sequence as set forth in SEQ ID NO:31, which corresponds to the sequence of the above indicated NCBI Reference Sequence of the DDX58 transcript, and also relates to the corresponding amino acid sequence for example as set forth in SEQ ID NO:32, which corresponds to the protein sequence defined in NCBI Protein Accession Reference Sequence NP_055129 encoding the DDX58 polypeptide.

The term “DDX58” also comprises nucleotide sequences showing a high degree of homology to DDX58, e.g., nucleic acid sequences being at least 75%, 80%, 85%, 90%, 91%, 92%, 93%, 94%, 95%, 96%, 97%, 98% or 99% identical to the sequence as set forth in SEQ ID NO:31 or amino acid sequences being at least 75%, 80%, 85%, 90%, 91%, 92%, 93%, 94%, 95%, 96%, 97%, 98% or 99% identical to the sequence as set forth in SEQ ID NO:32 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:32 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:31.

The term “DHX9” refers to the DExD/H-box Helicase 9 gene (Ensembl: ENSG00000135829), for example, to the sequence as defined in NCBI Reference Sequence NM_001357, specifically, to the nucleotide sequence as set forth in SEQ ID NO:33, which corresponds to the sequence of the above indicated NCBI Reference Sequence of the DHX9 transcript, and also relates to the corresponding amino acid sequence for example as set forth in SEQ ID NO:34, which corresponds to the protein sequence defined in NCBI Protein Accession Reference Sequence NP_001348 encoding the DHX9 polypeptide.

The term “DHX9” also comprises nucleotide sequences showing a high degree of homology to DHX9, e.g., nucleic acid sequences being at least 75%, 80%, 85%, 90%, 91%, 92%, 93%, 94%, 95%, 96%, 97%, 98% or 99% identical to the sequence as set forth in SEQ ID NO:33 or amino acid sequences being at least 75%, 80%, 85%, 90%, 91%, 92%, 93%, 94%, 95%, 96%, 97%, 98% or 99% identical to the sequence as set forth in SEQ ID NO:34 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:34 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:33.

The term “EZR” refers to the Ezrin gene (Ensembl: ENSG00000092820), for example, to the sequence as defined in NCBI Reference Sequence NM_003379, specifically, to the nucleotide sequence as set forth in SEQ ID NO:35, which corresponds to the sequence of the above indicated NCBI Reference Sequence of the EZR transcript, and also relates to the corresponding amino acid sequence for example as set forth in SEQ ID NO:36, which corresponds to the protein sequence defined in NCBI Protein Accession Reference Sequence NP_003370 encoding the EZR polypeptide.

The term “EZR” also comprises nucleotide sequences showing a high degree of homology to EZR, e.g., nucleic acid sequences being at least 75%, 80%, 85%, 90%, 91%, 92%, 93%, 94%, 95%, 96%, 97%, 98% or 99% identical to the sequence as set forth in SEQ ID NO:35 or amino acid sequences being at least 75%, 80%, 85%, 90%, 91%, 92%, 93%, 94%, 95%, 96%, 97%, 98% or 99% identical to the sequence as set forth in SEQ ID NO:36 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:36 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:35.

The term “FYN” refers to the FYN Proto-Oncogene gene (Ensembl: ENSG00000010810), for example, to the sequence as defined in NCBI Reference Sequence NM_002037 or in NCBI Reference Sequence NM_153047 or in NCBI Reference Sequence NM_153048, specifically, to the nucleotide sequence as set forth in SEQ ID NO:37 or in SEQ ID NO:38 or in SEQ ID NO:39, which correspond to the sequences of the above indicated NCBI Reference Sequences of the FYN transcript, and also relates to the corresponding amino acid sequence for example as set forth in SEQ ID NO:40 or in SEQ ID NO:41 or in SEQ ID NO:42, which correspond to the protein sequences defined in NCBI Protein Accession Reference Sequence NP_002028 and in NCBI Protein Accession Reference Sequence NP_694592 and in NCBI Protein Accession Reference Sequence XP_005266949 encoding the FYN polypeptide.

The term “FYN” also comprises nucleotide sequences showing a high degree of homology to FYN, e.g., nucleic acid sequences being at least 75%, 80%, 85%, 90%, 91%, 92%, 93%, 94%, 95%, 96%, 97%, 98% or 99% identical to the sequence as set forth in SEQ ID NO:37 or in SEQ ID NO:38 or in SEQ ID NO:39 or amino acid sequences being at least 75%, 80%, 85%, 90%, 91%, 92%, 93%, 94%, 95%, 96%, 97%, 98% or 99% identical to the sequence as set forth in SEQ ID NO:40 or in SEQ ID NO:41 or in SEQ ID NO:42 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:40 or in SEQ ID NO:41 or in SEQ ID NO:42 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:37 or in SEQ ID NO:38 or in SEQ ID NO:39.

The term “IFI16” refers to the Interferon Gamma Inducible Protein 16 gene (Ensembl: ENSG00000163565), for example, to the sequence as defined in NCBI Reference Sequence NM_005531, specifically, to the nucleotide sequence as set forth in SEQ ID NO:43, which corresponds to the sequence of the above indicated NCBI Reference Sequence of the IF116 transcript, and also relates to the corresponding amino acid sequence for example as set forth in SEQ ID NO:44, which corresponds to the protein sequence defined in NCBI Protein Accession Reference Sequence NP_005522 encoding the IF116 polypeptide.

The term “IFI16” also comprises nucleotide sequences showing a high degree of homology to IF116, e.g., nucleic acid sequences being at least 75%, 80%, 85%, 90%, 91%, 92%, 93%, 94%, 95%, 96%, 97%, 98% or 99% identical to the sequence as set forth in SEQ ID NO:43 or amino acid sequences being at least 75%, 80%, 85%, 90%, 91%, 92%, 93%, 94%, 95%, 96%, 97%, 98% or 99% identical to the sequence as set forth in SEQ ID NO:44 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:44 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:43.

The term “IFIH1” refers to the Interferon Induced With Helicase C Domain 1 gene (Ensembl: ENSG00000115267), for example, to the sequence as defined in NCBI Reference Sequence NM_022168, specifically, to the nucleotide sequence as set forth in SEQ ID NO:45, which corresponds to the sequence of the above indicated NCBI Reference Sequence of the IFIH1 transcript, and also relates to the corresponding amino acid sequence for example as set forth in SEQ ID NO:46, which corresponds to the protein sequence defined in NCBI Protein Accession Reference Sequence NP_071451 encoding the IFIH1 polypeptide.

The term “IFIH1” also comprises nucleotide sequences showing a high degree of homology to IFIH1, e.g., nucleic acid sequences being at least 75%, 80%, 85%, 90%, 91%, 92%, 93%, 94%, 95%, 96%, 97%, 98% or 99% identical to the sequence as set forth in SEQ ID NO:45 or amino acid sequences being at least 75%, 80%, 85%, 90%, 91%, 92%, 93%, 94%, 95%, 96%, 97%, 98% or 99% identical to the sequence as set forth in SEQ ID NO:46 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:46 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:45.

The term “IFIT1” refers to the Interferon Induced Protein With Tetratricopeptide Repeats 1 gene (Ensembl: ENSG00000185745), for example, to the sequence as defined in NCBI Reference Sequence NM_001270929 or in NCBI Reference Sequence NM_001548.5, specifically, to the nucleotide sequence as set forth in SEQ ID NO:47 or in SEQ ID NO:48, which correspond to the sequences of the above indicated NCBI Reference Sequences of the IFIT1 transcript, and also relates to the corresponding amino acid sequence for example as set forth in SEQ ID NO:49 or in SEQ ID NO:50, which correspond to the protein sequences defined in NCBI Protein Accession Reference Sequence NP_001257858 and in NCBI Protein Accession Reference Sequence NP_001539 encoding the IFIT1 polypeptide.

The term “IFIT1” also comprises nucleotide sequences showing a high degree of homology to IFIT1, e.g., nucleic acid sequences being at least 75%, 80%, 85%, 90%, 91%, 92%, 93%, 94%, 95%, 96%, 97%, 98% or 99% identical to the sequence as set forth in SEQ ID NO:47 or in SEQ ID NO:48 or amino acid sequences being at least 75%, 80%, 85%, 90%, 91%, 92%, 93%, 94%, 95%, 96%, 97%, 98% or 99% identical to the sequence as set forth in SEQ ID NO:49 or in SEQ ID NO:50 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:49 or SEQ ID NO:50 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:47 or in SEQ ID NO:48.

The term “IFIT3” refers to the Interferon Induced Protein With Tetratricopeptide Repeats 3 gene (Ensembl: ENSG00000119917), for example, to the sequence as defined in NCBI Reference Sequence NM_001031683, specifically, to the nucleotide sequence as set forth in SEQ ID NO:51, which corresponds to the sequence of the above indicated NCBI Reference Sequence of the IFIT3 transcript, and also relates to the corresponding amino acid sequence for example as set forth in SEQ ID NO:52, which corresponds to the protein sequence defined in NCBI Protein Accession Reference Sequence NP_001026853 encoding the IFIT3 polypeptide.

The term “IFIT3” also comprises nucleotide sequences showing a high degree of homology to IFIT3, e.g., nucleic acid sequences being at least 75%, 80%, 85%, 90%, 91%, 92%, 93%, 94%, 95%, 96%, 97%, 98% or 99% identical to the sequence as set forth in SEQ ID NO:51 or amino acid sequences being at least 75%, 80%, 85%, 90%, 91%, 92%, 93%, 94%, 95%, 96%, 97%, 98% or 99% identical to the sequence as set forth in SEQ ID NO:52 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:52 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:51.

The term “KIAA1549” refers to the human KIAA1549 gene (Ensembl: ENSG00000122778), for example, to the sequence as defined in NCBI Reference Sequence NM_020910 or in NCBI Reference Sequence NM_001164665, specifically, to the nucleotide sequence as set forth in SEQ ID NO:53 or in SEQ ID NO:54, which correspond to the sequences of the above indicated NCBI Reference Sequence of the KIAA1549 transcript, and also relates to the corresponding amino acid sequence for example as set forth in SEQ ID NO:55 or in SEQ ID NO:56, which correspond to the protein sequences defined in NCBI Protein Accession Reference Sequence NP_065961 and in NCBI Protein Accession Reference Sequence NP_001158137 encoding the KIAA1549 polypeptide.

The term “KIAA1549” also comprises nucleotide sequences showing a high degree of homology to KIAA1549, e.g., nucleic acid sequences being at least 75%, 80%, 85%, 90%, 91%, 92%, 93%, 94%, 95%, 96%, 97%, 98% or 99% identical to the sequence as set forth in SEQ ID NO:53 or in SEQ ID NO:54 or amino acid sequences being at least 75%, 80%, 85%, 90%, 91%, 92%, 93%, 94%, 95%, 96%, 97%, 98% or 99% identical to the sequence as set forth in SEQ ID NO:55 or in SEQ ID NO:56 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:55 or in SEQ ID NO:56 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:53 or in SEQ ID NO:54.

The term “LAT” refers to the Linker For Activation Of T-Cells gene (Ensembl: ENSG00000213658), for example, to the sequence as defined in NCBI Reference Sequence NM_001014987 or in NCBI Reference Sequence NM_014387, specifically, to the nucleotide sequence as set forth in SEQ ID NO:57 or in SEQ ID NO:58, which correspond to the sequences of the above indicated NCBI Reference Sequences of the LAT transcript, and also relates to the corresponding amino acid sequence for example as set forth in SEQ ID NO:59 or in SEQ ID NO:60, which corresponds to the protein sequences defined in NCBI Protein Accession Reference Sequence NP_001014987 and in NCBI Protein Accession Reference Sequence NP_055202 encoding the LAT polypeptide.

The term “LAT” also comprises nucleotide sequences showing a high degree of homology to LAT, e.g., nucleic acid sequences being at least 75%, 80%, 85%, 90%, 91%, 92%, 93%, 94%, 95%, 96%, 97%, 98% or 99% identical to the sequence as set forth in SEQ ID NO:57 or in SEQ ID NO:58 or amino acid sequences being at least 75%, 80%, 85%, 90%, 91%, 92%, 93%, 94%, 95%, 96%, 97%, 98% or 99% identical to the sequence as set forth in SEQ ID NO:59 or in SEQ ID NO:60 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:59 or in SEQ ID NO:60 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:57 or in SEQ ID NO:58.

The term “LCK” refers to the LCK Proto-Oncogene gene (Ensembl: ENSG00000182866), for example, to the sequence as defined in NCBI Reference Sequence NM_005356, specifically, to the nucleotide sequence as set forth in SEQ ID NO:61, which corresponds to the sequence of the above indicated NCBI Reference Sequence of the LCK transcript, and also relates to the corresponding amino acid sequence for example as set forth in SEQ ID NO:62, which corresponds to the protein sequence defined in NCBI Protein Accession Reference Sequence NP_005347 encoding the LCK polypeptide.

The term “LCK” also comprises nucleotide sequences showing a high degree of homology to LCK, e.g., nucleic acid sequences being at least 75%, 80%, 85%, 90%, 91%, 92%, 93%, 94%, 95%, 96%, 97%, 98% or 99% identical to the sequence as set forth in SEQ ID NO:61 or amino acid sequences being at least 75%, 80%, 85%, 90%, 91%, 92%, 93%, 94%, 95%, 96%, 97%, 98% or 99% identical to the sequence as set forth in SEQ ID NO:62 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:62 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:61.

The term “LRRFIP1” refers to the LRR Binding FLII Interacting Protein 1 gene (Ensembl: ENSG00000124831), for example, to the sequence as defined in NCBI Reference Sequence NM_004735 or in NCBI Reference Sequence NM_001137550 or in NCBI Reference Sequence NM_001137553 or in NCBI Reference Sequence NM_001137552, specifically, to the nucleotide sequence as set forth in SEQ ID NO:63 or in SEQ ID NO:64 or in SEQ ID NO:65 or in SEQ ID NO:66, which correspond to the sequences of the above indicated NCBI Reference Sequences of the LRRFIP1 transcript, and also relates to the corresponding amino acid sequence for example as set forth in SEQ ID NO:67 or in SEQ ID NO:68 or in SEQ ID NO:69 or in SEQ ID NO:70, which correspond to the protein sequences defined in NCBI Protein Accession Reference Sequence NP_004726 and in NCBI Protein Accession Reference Sequence NP_001131022 and in NCBI Protein Accession Reference Sequence NP_001131025 and in NCBI Protein Accession Reference Sequence NP_001131024 encoding the LRRFIP1 polypeptide.

The term “LRRFIP1” also comprises nucleotide sequences showing a high degree of homology to LRRFIP1, e.g., nucleic acid sequences being at least 75%, 80%, 85%, 90%, 91%, 92%, 93%, 94%, 95%, 96%, 97%, 98% or 99% identical to the sequence as set forth in SEQ ID NO:63 or in SEQ ID NO:64 or in SEQ ID NO:65 or in SEQ ID NO:66 or amino acid sequences being at least 75%, 80%, 85%, 90%, 91%, 92%, 93%, 94%, 95%, 96%, 97%, 98% or 99% identical to the sequence as set forth in SEQ ID NO:67 or in SEQ ID NO:68 or in SEQ ID NO:69 or in SEQ ID NO:70 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:67 or in SEQ ID NO:68 or in SEQ ID NO:69 or in SEQ ID NO:70 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:63 or in SEQ ID NO:64 or in SEQ ID NO:65 or in SEQ ID NO:66.

The term “MYD88” refers to the MYD88 Innate Immune Signal Transduction Adaptor gene (Ensembl: ENSG00000172936), for example, to the sequence as defined in NCBI Reference Sequence NM_001172567 or in NCBI Reference Sequence NM_001172568 or in NCBI Reference Sequence NM_001172569 or in NCBI Reference Sequence NM_001172566 or in NCBI Reference Sequence NM_002468, specifically, to the nucleotide sequences as set forth in SEQ ID NO:71 or in SEQ ID NO:72 or in SEQ ID NO:73 or in SEQ ID NO:74 or in SEQ ID NO:75, which correspond to the sequences of the above indicated NCBI Reference Sequences of the MYD88 transcript, and also relates to the corresponding amino acid sequence for example as set forth in SEQ ID NO:76 or in SEQ ID NO:77 or in SEQ ID NO:78 or in SEQ ID NO:79 or in SEQ ID NO:80, which correspond to the protein sequences defined in NCBI Protein Accession Reference Sequence NP_001166038 and in NCBI Protein Accession Reference Sequence NP_001166039 and in NCBI Protein Accession Reference Sequence NP_001166040 and in NCBI Protein Accession Reference Sequence NP_001166037 and in NCBI Protein Accession Reference Sequence NP_002459 encoding the MYD88 polypeptide.

The term “MYD88” also comprises nucleotide sequences showing a high degree of homology to MYD88, e.g., nucleic acid sequences being at least 75%, 80%, 85%, 90%, 91%, 92%, 93%, 94%, 95%, 96%, 97%, 98% or 99% identical to the sequence as set forth in SEQ ID NO:71 or in SEQ ID NO:72 or in SEQ ID NO:73 or in SEQ ID NO:74 or in SEQ ID NO:75 or amino acid sequences being at least 75%, 80%, 85%, 90%, 91%, 92%, 93%, 94%, 95%, 96%, 97%, 98% or 99% identical to the sequence as set forth in SEQ ID NO:76 or in SEQ ID NO:77 or in SEQ ID NO:78 or in SEQ ID NO:79 or in SEQ ID NO:80 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:76 or in SEQ ID NO:77 or in SEQ ID NO:78 or in SEQ ID NO:79 or in SEQ ID NO:80 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:71 or in SEQ ID NO:72 or in SEQ ID NO:73 or in SEQ ID NO:74 or in SEQ ID NO:75.

The term “OAS1” refers to the 2′-5′-Oligoadenylate Synthetase 1 gene (Ensembl: ENSG00000089127), for example, to the sequence as defined in NCBI Reference Sequence NM_001320151 or in NCBI Reference Sequence NM_002534 or in NCBI Reference Sequence NM_001032409 or in NCBI Reference Sequence NM_016816, specifically, to the nucleotide sequences as set forth in SEQ ID NO:81 or in SEQ ID NO:82 or in SEQ ID NO:83 or in SEQ ID NO:84, which correspond to the sequences of the above indicated NCBI Reference Sequences of the OAS1 transcript, and also relates to the corresponding amino acid sequences for example as set forth in SEQ ID NO:85 or in SEQ ID NO:86 or in SEQ ID NO:87 or in SEQ ID NO:88, which correspond to the protein sequences defined in NCBI Protein Accession Reference Sequence NP_001307080 and in NCBI Protein Accession Reference Sequence NP_002525 and in NCBI Protein Accession Reference Sequence NP_001027581 and in NCBI Protein Accession Reference Sequence NP_058132 encoding the OAS1 polypeptide.

The term “OAS1” also comprises nucleotide sequences showing a high degree of homology to OAS1, e.g., nucleic acid sequences being at least 75%, 80%, 85%, 90%, 91%, 92%, 93%, 94%, 95%, 96%, 97%, 98% or 99% identical to the sequence as set forth in SEQ ID NO:81 or in SEQ ID NO:82 or in SEQ ID NO:83 or in SEQ ID NO:84 or amino acid sequences being at least 75%, 80%, 85%, 90%, 91%, 92%, 93%, 94%, 95%, 96%, 97%, 98% or 99% identical to the sequence as set forth in SEQ ID NO:85 or in SEQ ID NO:86 or in SEQ ID NO:87 or in SEQ ID NO:88 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:85 or in SEQ ID NO:86 or in SEQ ID NO:87 or in SEQ ID NO:88 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:81 or in SEQ ID NO:82 or in SEQ ID NO:83 or in SEQ ID NO:84.

The term “PAG1” refers to the Phosphoprotein Membrane Anchor With Glycosphingolipid Microdomains 1 gene (Ensembl: ENSG00000076641), for example, to the sequence as defined in NCBI Reference Sequence NM_018440, specifically, to the nucleotide sequence as set forth in SEQ ID NO:89, which corresponds to the sequence of the above indicated NCBI Reference Sequence of the PAG1 transcript, and also relates to the corresponding amino acid sequence for example as set forth in SEQ ID NO:90, which corresponds to the protein sequence defined in NCBI Protein Accession Reference Sequence NP_060910 encoding the PAG1 polypeptide.

The term “PAG1” also comprises nucleotide sequences showing a high degree of homology to PAG1, e.g., nucleic acid sequences being at least 75%, 80%, 85%, 90%, 91%, 92%, 93%, 94%, 95%, 96%, 97%, 98% or 99% identical to the sequence as set forth in SEQ ID NO:89 or amino acid sequences being at least 75%, 80%, 85%, 90%, 91%, 92%, 93%, 94%, 95%, 96%, 97%, 98% or 99% identical to the sequence as set forth in SEQ ID NO:90 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:90 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:89.

The term “PDE4D” refers to the human Phosphodiesterase 4D gene (Ensembl: ENSG00000113448), for example, to the sequence as defined in NCBI Reference Sequence NM_001104631 or in NCBI Reference Sequence NM_001349242 or in NCBI Reference Sequence NM_001197218 or in NCBI Reference Sequence NM_006203 or in NCBI Reference Sequence NM_001197221 or in NCBI Reference Sequence NM_001197220 or in NCBI Reference Sequence NM_001197223 or in NCBI Reference Sequence NM_001165899 or in NCBI Reference Sequence NM_001165899, specifically, to the nucleotide sequence as set forth in SEQ ID NO:91 or in SEQ ID NO:92 or in SEQ ID NO:93 or in SEQ ID NO:94 or in SEQ ID NO:95 or in SEQ ID NO:96 or in SEQ ID NO:97 or in SEQ ID NO:98 or in SEQ ID NO:99, which correspond to the sequences of the above indicated NCBI Reference Sequence of the PDE4D transcript, and also relates to the corresponding amino acid sequence for example as set forth in SEQ ID NO:100 or in SEQ ID NO:101 or in SEQ ID NO:102 or in SEQ ID NO:103 or in SEQ ID NO:104 or in SEQ ID NO:105 or in SEQ ID NO:106 or in SEQ ID NO:107 or in SEQ ID NO:108, which correspond to the protein sequences defined in NCBI Protein Accession Reference Sequence NP_001098101 and in NCBI Protein Accession Reference Sequence NP_001336171 and in NCBI Protein Accession Reference Sequence NP_001184147 and in NCBI Protein Accession Reference Sequence NP_006194 and in NCBI Protein Accession Reference Sequence NP_001184150 and in NCBI Protein Accession Reference Sequence NP_001184149 and in NCBI Protein Accession Reference Sequence NP_001184152 and in NCBI Protein Accession Reference Sequence NP_001159371 and in NCBI Protein Accession Reference Sequence NP_001184148 encoding the PDE4D polypeptide.

The term “PDE4D” also comprises nucleotide sequences showing a high degree of homology to PDE4D, e.g., nucleic acid sequences being at least 75%, 80%, 85%, 90%, 91%, 92%, 93%, 94%, 95%, 96%, 97%, 98% or 99% identical to the sequence as set forth in SEQ ID NO:91 or in SEQ ID NO:92 or in SEQ ID NO:93 or in SEQ ID NO:94 or in SEQ ID NO:95 or in SEQ ID NO:96 or in SEQ ID NO:97 or in SEQ ID NO:98 or in SEQ ID NO:99 or amino acid sequences being at least 75%, 80%, 85%, 90%, 91%, 92%, 93%, 94%, 95%, 96%, 97%, 98% or 99% identical to the sequence as set forth in SEQ ID NO:100 or in SEQ ID NO:101 or in SEQ ID NO:102 or in SEQ ID NO:103 or in SEQ ID NO:104 or in SEQ ID NO:105 or in SEQ ID NO:106 or in SEQ ID NO:107 or in SEQ ID NO:108 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:100 or in SEQ ID NO:101 or in SEQ ID NO:102 or in SEQ ID NO:103 or in SEQ ID NO:104 or in SEQ ID NO:105 or in SEQ ID NO:106 or in SEQ ID NO:107 or in SEQ ID NO:108 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:91 or in SEQ ID NO:92 or in SEQ ID NO:93 or in SEQ ID NO:94 or in SEQ ID NO:95 or in SEQ ID NO:96 or in SEQ ID NO:97 or in SEQ ID NO:98 or in SEQ ID NO:99.

The term “PRKACA” refers to the Protein Kinase cAMP-Activated Catalytic Subunit Alpha gene (Ensembl: ENSG00000072062), for example, to the sequence as defined in NCBI Reference Sequence NM_002730 or in NCBI Reference Sequence NM_207518, specifically, to the nucleotide sequences as set forth in SEQ ID NO:109 or in SEQ ID NO:110, which correspond to the sequences of the above indicated NCBI Reference Sequences of the PRKACA transcript, and also relates to the corresponding amino acid sequence for example as set forth in SEQ ID NO:111 or in SEQ ID NO:112, which correspond to the protein sequences defined in NCBI Protein Accession Reference Sequence NP_002721 and in NCBI Protein Accession Reference Sequence NP_997401 encoding the PRKACA polypeptide.

The term “PRKACA” also comprises nucleotide sequences showing a high degree of homology to PRKACA, e.g., nucleic acid sequences being at least 75%, 80%, 85%, 90%, 91%, 92%, 93%, 94%, 95%, 96%, 97%, 98% or 99% identical to the sequence as set forth in SEQ ID NO:109 or in SEQ ID NO:110 or amino acid sequences being at least 75%, 80%, 85%, 90%, 91%, 92%, 93%, 94%, 95%, 96%, 97%, 98% or 99% identical to the sequence as set forth in SEQ ID NO:111 or in SEQ ID NO:112 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:111 or in SEQ ID NO:112 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:109 or in SEQ ID NO:110.

The term “PRKACB” refers to the Protein Kinase cAMP-Activated Catalytic Subunit Beta gene (Ensembl: ENSG00000142875), for example, to the sequence as defined in NCBI Reference Sequence NM_002731 or in NCBI Reference Sequence NM_182948 or in NCBI Reference Sequence NM_001242860 or in NCBI Reference Sequence NM_001242859 or in NCBI Reference Sequence NM_001242858 or in NCBI Reference Sequence NM_001242862 or in NCBI Reference Sequence NM_001242861 or in NCBI Reference Sequence NM_001300915 or in NCBI Reference Sequence NM_207578 or in NCBI Reference Sequence NM_001242857 or in NCBI Reference Sequence NM_001300917, specifically, to the nucleotide sequence as set forth in SEQ ID NO:113 or in SEQ ID NO:114 or in SEQ ID NO:115 or in SEQ ID NO:116 or in SEQ ID NO:117 or in SEQ ID NO:118 or in SEQ ID NO:119 or in SEQ ID NO:120 or in SEQ ID NO:121 or in SEQ ID NO:122 or in SEQ ID NO:123, which correspond to the sequences of the above indicated NCBI Reference Sequences of the PRKACB transcript, and also relates to the corresponding amino acid sequence for example as set forth in SEQ ID NO:124 or in SEQ ID NO:125 or in SEQ ID NO:126 or in SEQ ID NO:127 or in SEQ ID NO:128 or in SEQ ID NO:129 or in SEQ ID NO:130 or in SEQ ID NO:131 or in SEQ ID NO:132 or in SEQ ID NO:133 or in SEQ ID NO:134, which correspond to the protein sequences defined in NCBI Protein Accession Reference Sequence NP_002722 and in NCBI Protein Accession Reference Sequence NP_891993 and in NCBI Protein Accession Reference Sequence NP_001229789 and in NCBI Protein Accession Reference Sequence NP_001229788 and in NCBI Protein Accession Reference Sequence NP_001229787 and in NCBI Protein Accession Reference Sequence NP_001229791 and in NCBI Protein Accession Reference Sequence NP_001229790 and in NCBI Protein Accession Reference Sequence NP_001287844 and in NCBI Protein Accession Reference Sequence NP_997461 and in NCBI Protein Accession Reference Sequence NP_001229786 and in NCBI Protein Accession Reference Sequence NP_001287846 encoding the PRKACB polypeptide.

The term “PRKACB” also comprises nucleotide sequences showing a high degree of homology to PRKACB, e.g., nucleic acid sequences being at least 75%, 80%, 85%, 90%, 91%, 92%, 93%, 94%, 95%, 96%, 97%, 98% or 99% identical to the sequence as set forth in SEQ ID NO:113 or in SEQ ID NO: 114 or in SEQ ID NO: 115 or in SEQ ID NO:116 or in SEQ ID NO:117 or in SEQ ID NO:118 or in SEQ ID NO:119 or in SEQ ID NO:120 or in SEQ ID NO:121 or in SEQ ID NO:122 or in SEQ ID NO:123 or amino acid sequences being at least 75%, 80%, 85%, 90%, 91%, 92%, 93%, 94%, 95%, 96%, 97%, 98% or 99% identical to the sequence as set forth in SEQ ID NO:124 or in SEQ ID NO:125 or in SEQ ID NO:126 or in SEQ ID NO:127 or in SEQ ID NO:128 or in SEQ ID NO:129 or in SEQ ID NO: 130 or in SEQ ID NO:131 or in SEQ ID NO:132 or in SEQ ID NO:133 or in SEQ ID NO:134 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:124 or in SEQ ID NO:125 or in SEQ ID NO:126 or in SEQ ID NO:127 or in SEQ ID NO:128 or in SEQ ID NO:129 or in SEQ ID NO:130 or in SEQ ID NO:131 or in SEQ ID NO:132 or in SEQ ID NO:133 or in SEQ ID NO:134 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:113 or in SEQ ID NO:114 or in SEQ ID NO:115 or in SEQ ID NO:116 or in SEQ ID NO:117 or in SEQ ID NO:118 or in SEQ ID NO:119 or in SEQ ID NO:120 or in SEQ ID NO:121 or in SEQ ID NO:122 or in SEQ ID NO:123.

The term “PTPRC” refers to the Protein Tyrosine Phosphatase Receptor Type C gene (Ensembl: ENSG00000081237), for example, to the sequence as defined in NCBI Reference Sequence NM_002838 or in NCBI Reference Sequence NM_080921, specifically, to the nucleotide sequence as set forth in SEQ ID NO:135 or in SEQ ID NO:136, which correspond to the sequences of the above indicated NCBI Reference Sequences of the PTPRC transcript, and also relates to the corresponding amino acid sequence for example as set forth in SEQ ID NO:137 or in SEQ ID NO:138, which correspond to the protein sequences defined in NCBI Protein Accession Reference Sequence NP_002829 encoding the PTPRC polypeptide and in NCBI Protein Accession Reference Sequence NP_563578.

The term “PTPRC” also comprises nucleotide sequences showing a high degree of homology to PTPRC, e.g., nucleic acid sequences being at least 75%, 80%, 85%, 90%, 91%, 92%, 93%, 94%, 95%, 96%, 97%, 98% or 99% identical to the sequence as set forth in SEQ ID NO:135 or in SEQ ID NO:136 or amino acid sequences being at least 75%, 80%, 85%, 90%, 91%, 92%, 93%, 94%, 95%, 96%, 97%, 98% or 99% identical to the sequence as set forth in SEQ ID NO:137 or in SEQ ID NO:138 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:137 or in SEQ ID NO:138 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:135 or in SEQ ID NO:136.

The term “RAP1GAP2” refers to the human RAP1 GTPase Activating Protein 2 gene (ENSG00000132359), for example, to the sequence as defined in NCBI Reference Sequence NM_015085 or in NCBI Reference Sequence NM_001100398 or in NCBI Reference Sequence NM_001330058, specifically, to the nucleotide sequence as set forth in SEQ ID NO:139 or in SEQ ID NO:140 or in SEQ ID NO:141, which correspond to the sequences of the above indicated NCBI Reference Sequences of the RAP1GAP2 transcript, and also relates to the corresponding amino acid sequence for example as set forth in SEQ ID NO:142 or in SEQ ID NO:143 or in SEQ ID NO:144, which correspond to the protein sequences defined in NCBI Protein Accession Reference Sequence NP_055900 and in NCBI Protein Accession Reference Sequence NP_001093868 and in NCBI Protein Accession Reference Sequence NP_001316987 encoding the RAP1GAP2 polypeptide.

The term “RAP1GAP2” also comprises nucleotide sequences showing a high degree of homology to RAP1GAP2, e.g., nucleic acid sequences being at least 75%, 80%, 85%, 90%, 91%, 92%, 93%, 94%, 95%, 96%, 97%, 98% or 99% identical to the sequence as set forth in SEQ ID NO:139 or in SEQ ID NO:140 or in SEQ ID NO:141 or amino acid sequences being at least 75%, 80%, 85%, 90%, 91%, 92%, 93%, 94%, 95%, 96%, 97%, 98% or 99% identical to the sequence as set forth in SEQ ID NO:142 or in SEQ ID NO:143 or in SEQ ID NO:144 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:142 or in SEQ ID NO:143 or in SEQ ID NO:144 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:139 or in SEQ ID NO:140 or in SEQ ID NO:141.

The term “SLC39A11” refers to the human Solute Carrier Family 39 Member 11 gene (Ensembl: ENSG00000133195), for example, to the sequence as defined in NCBI Reference Sequence NM_139177 or in NCBI Reference Sequence NM_001352692, specifically, to the nucleotide sequence as set forth in SEQ ID NO:145 or in SEQ ID NO:146, which correspond to the sequences of the above indicated NCBI Reference Sequences of the SLC39A11 transcript, and also relates to the corresponding amino acid sequence for example as set forth in SEQ ID NO:147 or in SEQ ID NO:148, which correspond to the protein sequences defined in NCBI Protein Accession Reference Sequence NP_631916 and in NCBI Protein Accession Reference Sequence NP_001339621 encoding the SLC39A11 polypeptide.

The term “SLC39A11” also comprises nucleotide sequences showing a high degree of homology to SLC39A11, e.g., nucleic acid sequences being at least 75%, 80%, 85%, 90%, 91%, 92%, 93%, 94%, 95%, 96%, 97%, 98% or 99% identical to the sequence as set forth in SEQ ID NO:145 or in SEQ ID NO:146 or amino acid sequences being at least 75%, 80%, 85%, 90%, 91%, 92%, 93%, 94%, 95%, 96%, 97%, 98% or 99% identical to the sequence as set forth in SEQ ID NO:147 or in SEQ ID NO:148 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:147 or in SEQ ID NO:148 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:145 or in SEQ ID NO:146.

The term “TDRD1” refers to the human Tudor Domain Containing 1 gene (Ensembl: ENSG00000095627), for example, to the sequence as defined in NCBI Reference Sequence NM_198795, specifically, to the nucleotide sequence as set forth in SEQ ID NO:149, which corresponds to the sequence of the above indicated NCBI Reference Sequence of the TDRD1 transcript, and also relates to the corresponding amino acid sequence for example as set forth in SEQ ID NO:150, which corresponds to the protein sequence defined in NCBI Protein Accession Reference Sequence NP_942090 encoding the TDRD1 polypeptide.

The term “TDRD1” also comprises nucleotide sequences showing a high degree of homology to TDRD1, e.g., nucleic acid sequences being at least 75%, 80%, 85%, 90%, 91%, 92%, 93%, 94%, 95%, 96%, 97%, 98% or 99% identical to the sequence as set forth in SEQ ID NO:149 or amino acid sequences being at least 75%, 80%, 85%, 90%, 91%, 92%, 93%, 94%, 95%, 96%, 97%, 98% or 99% identical to the sequence as set forth in SEQ ID NO:150 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:150 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:149.

The term “TLR8” refers to the Toll Like Receptor 8 gene (Ensembl: ENSG00000101916), for example, to the sequence as defined in NCBI Reference Sequence NM_138636 or in NCBI Reference Sequence NM_016610, specifically, to the nucleotide sequences as set forth in SEQ ID NO:151 or in SEQ ID NO:152, which correspond to the sequences of the above indicated NCBI Reference Sequences of the TLR8 transcript, and also relates to the corresponding amino acid sequences for example as set forth in SEQ ID NO:153 or in SEQ ID NO:154, which corresponds to the protein sequences defined in NCBI Protein Accession Reference Sequence NP_619542 and in NCBI Protein Accession Reference Sequence NP_057694 encoding the TLR8 polypeptide.

The term “TLR8” also comprises nucleotide sequences showing a high degree of homology to TLR8, e.g., nucleic acid sequences being at least 75%, 80%, 85%, 90%, 91%, 92%, 93%, 94%, 95%, 96%, 97%, 98% or 99% identical to the sequence as set forth in SEQ ID NO:151 or in SEQ ID NO:152 or amino acid sequences being at least 75%, 80%, 85%, 90%, 91%, 92%, 93%, 94%, 95%, 96%, 97%, 98% or 99% identical to the sequence as set forth in SEQ ID NO:153 or in SEQ ID NO:154 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:153 or in SEQ ID NO:154 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:151 or in SEQ ID NO:152.

The term “VWA2” refers to the human Von Willebrand Factor A Domain Containing 2 gene (Ensembl: ENSG00000165816), for example, to the sequence as defined in NCBI Reference Sequence NM_001320804, specifically, to the nucleotide sequence as set forth in SEQ ID NO:155, which corresponds to the sequence of the above indicated NCBI Reference Sequence of the VWA2 transcript, and also relates to the corresponding amino acid sequence for example as set forth in SEQ ID NO:156, which corresponds to the protein sequence defined in NCBI Protein Accession Reference Sequence NP_001307733 encoding the VWA2 polypeptide.

The term “VWA2” also comprises nucleotide sequences showing a high degree of homology to VWA2, e.g., nucleic acid sequences being at least 75%, 80%, 85%, 90%, 91%, 92%, 93%, 94%, 95%, 96%, 97%, 98% or 99% identical to the sequence as set forth in SEQ ID NO:155 or amino acid sequences being at least 75%, 80%, 85%, 90%, 91%, 92%, 93%, 94%, 95%, 96%, 97%, 98% or 99% identical to the sequence as set forth in SEQ ID NO:156 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:156 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:155.

The term “ZAP70” refers to the Zeta Chain Of T-Cell Receptor Associated Protein Kinase 70 gene (Ensembl: ENSG00000115085), for example, to the sequence as defined in NCBI Reference Sequence NM_001079 or in NCBI Reference Sequence NM 207519, specifically, to the nucleotide sequences as set forth in SEQ ID NO:157 or in SEQ ID NO:158, which correspond to the sequences of the above indicated NCBI Reference Sequences of the ZAP70 transcript, and also relates to the corresponding amino acid sequence for example as set forth in SEQ ID NO:159 or in SEQ ID NO:160, which corresponds to the protein sequences defined in NCBI Protein Accession Reference Sequence NP_001070 and in NCBI Protein Accession Reference Sequence NP_997402 encoding the ZAP70 polypeptide.

The term “ZAP70” also comprises nucleotide sequences showing a high degree of homology to ZAP70, e.g., nucleic acid sequences being at least 75%, 80%, 85%, 90%, 91%, 92%, 93%, 94%, 95%, 96%, 97%, 98% or 99% identical to the sequence as set forth in SEQ ID NO:157 or in SEQ ID NO:158 or amino acid sequences being at least 75%, 80%, 85%, 90%, 91%, 92%, 93%, 94%, 95%, 96%, 97%, 98% or 99% identical to the sequence as set forth in SEQ ID NO:159 or in SEQ ID NO:160 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:159 or in SEQ ID NO:160 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:157 or in SEQ ID NO:158.

The term “ZBP1” refers to the Z-DNA Binding Protein 1 gene (Ensembl: ENSG00000124256), for example, to the sequence as defined in NCBI Reference Sequence NM_030776 or in NCBI Reference Sequence NM_001160418 or in NCBI Reference Sequence NM_001160419, specifically, to the nucleotide sequence as set forth in SEQ ID NO:161 or in SEQ ID NO:162 or in SEQ ID NO:163, which corresponds to the sequence of the above indicated NCBI Reference Sequence of the ZBP1 transcript, and also relates to the corresponding amino acid sequence for example as set forth in SEQ ID NO:164 or in SEQ ID NO:165 or in SEQ ID NO:166, which corresponds to the protein sequences defined in NCBI Protein Accession Reference Sequence NP_110403 and in NCBI Protein Accession Reference Sequence NP_001153890 and in NCBI Protein Accession Reference Sequence NP_001153891 encoding the ZBP1 polypeptide.

The term “ZBP1” also comprises nucleotide sequences showing a high degree of homology to ZBP1, e.g., nucleic acid sequences being at least 75%, 80%, 85%, 90%, 91%, 92%, 93%, 94%, 95%, 96%, 97%, 98% or 99% identical to the sequence as set forth in SEQ ID NO:161 or in SEQ ID NO:162 or in SEQ ID NO:163 or amino acid sequences being at least 75%, 80%, 85%, 90%, 91%, 92%, 93%, 94%, 95%, 96%, 97%, 98% or 99% identical to the sequence as set forth in SEQ ID NO:164 or in SEQ ID NO:165 or in SEQ ID NO:166 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:164 or in SEQ ID NO:165 or in SEQ ID NO:166 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:161 or in SEQ ID NO:162 or in SEQ ID NO:163.

The term “biological sample” or “sample obtained from a subject” refers to any biological material obtained via suitable methods known to the person skilled in the art from a subject, e.g., a colorectal cancer patient. The biological sample used may be collected in a clinically acceptable manner, e.g., in a way that nucleic acids (in particular RNA) or proteins are preserved.

The biological sample(s) may include body tissue and/or a fluid, such as, but not limited to, blood (or blood derived such as serum, plasma, or PBMC's (peripheral blood mononuclear cells)), sweat, saliva, urine, and a needle biopsy or resection biopsy. Furthermore, the biological sample may contain a cell extract derived from or a cell population including an epithelial cell, such as a cancerous epithelial cell or an epithelial cell derived from tissue suspected to be cancerous. The biological sample may contain a cell population derived from a glandular tissue, e.g., the sample may be derived from the colorectal of a subject. Additionally, cells may be purified from obtained body tissues and fluids if necessary, and then used as the biological sample. In some realizations, the sample may be a tissue sample, a urine sample, a urine sediment sample, a blood sample, a saliva sample, a semen sample, a sample including circulating tumour cells, extracellular vesicles, a sample containing prostate secreted exosomes, or cell lines or cancer cell line.

In one particular realization, biopsy or resections samples may be obtained and/or used. Such samples may include cells or cell lysates.

It is also conceivable that the content of a biological sample is submitted to an enrichment step. For instance, a sample may be contacted with ligands specific for the cell membrane or organelles of certain cell types, e.g., colorectal cells, functionalized for example with magnetic particles. The material concentrated by the magnetic particles may subsequently be used for detection and analysis steps as described herein above or below.

Furthermore, cells, e.g., tumour cells, may be enriched via filtration processes of fluid or liquid samples, e.g., blood, urine, etc. Such filtration processes may also be combined with enrichment steps based on ligand specific interactions as described herein above.

The term “colorectal cancer” refers to a cancer of the colon or the rectum. Colorectal cancers start in either the colon or the rectum, which make up a large part of the intestine, which in turn is part of the gastrointestinal (GI) system. Colorectal cancers can also be called colon cancer or rectal cancer, depending on their site of origin. However, these cancers are often grouped together as colorectal cancer due to their common nature. The term “TNM” refers to a classification system, which is used to describe the characteristics of malignant tumors.

The term “T stage” refers to the extent and size of the tumor according to the TNM classification system. T stage can have the attributes T1 (small local tumor; typically <2 cm in size); T2 (larger local tumor; typically 2-5 cm in size); T3 (larger locally advanced tumor; typically >5 cm in size; T4 (advanced/metastatic tumor).

The term “N stage” refers to the presence and extent of tumor positive lymph nodes according to the TNM classification system. N stage can have the attributes NO (no evidence of tumor positive lymph nodes); N1 (evidence of tumor positive lymph nodes; N2/N3 (more extended number of tumor positive lymph nodes); NX (no assessment of lymph node status possible).

The term “M stage” refers to the presence and extent of tumor metastases according to the TNM classification system. M stage can have the attributes M0 (no evidence of the presence of distant metastases); M1 (evidence of the presence of distant metastases).

The term “survival” refers to survival of a patient from his or her colorectal cancer.

It is Preferred that:

the six or more gene expression levels comprise
one or more immune defense response genes, preferably two or more, more preferably three or more, most preferably all of the immune defense genes, and
one or more T-Cell receptor signaling genes, preferably two or more, more preferably three or more, most preferably all of the T-Cell receptor signaling genes, and
one or more PDE4D7 correlated genes, preferably two or more, more preferably three or more, most preferably all of the PDE4D7 correlated genes.

In an Embodiment:
the one or more immune defense response genes comprise three or more, preferably, six or more, more preferably, nine or more, most preferably, all of the immune defense genes, and/or
the one or more T-Cell receptor signaling genes comprise three or more, preferably, six or more, more preferably, nine or more, most preferably, all of the T-Cell receptor signaling genes, and/or
the one or more PDE4D7 correlated genes comprise three or more, preferably, six or more, most preferably, all of the PDE4D7 correlated genes.

It is preferred that the determining of the outcome comprises:
combining the first gene expression profiles for two or more, for example, 2, 3, 4, 5, 6, 7, 8, 9, 10, 11, 12, 13 or all, of the immune defense response genes with a regression function that had been derived from a population of colorectal cancer subjects, and/or
combining the second gene expression profiles for two or more, for example, 2, 3, 4, 5, 6, 7, 8, 9, 10, 11, 12, 13, 14, 15, 16 or all, of the T-Cell receptor signaling genes with a regression function that had been derived from a population of colorectal cancer subjects, and/or
combining the third gene expression profiles for two or more, for example, 2, 3, 4, 5, 6, 7 or all, of the PDE4D7 correlated genes with a regression function that had been derived from a population of colorectal cancer subjects, and/or
combining the six or more gene expression levels, for example 6, 7, 8, 9, 10, 11, 12, 13, 14, 15, 16, or more, or all gene expression levels with a regression function that had been derived from a population of colorectal cancer subjects.

Cox proportional-hazards regression allows analyzing the effect of several risk factors on time to a tested event like survival. Thereby, the risk factors may be dichotomous or discrete variables like a risk score or a clinical stage but may also be a continuous variable like a biomarker measurement or gene expression values. The probability of the endpoint (e.g., death or disease recurrence) is called the hazard. Next to the information on whether or not the tested endpoint was reached by e.g. subject in a patient cohort (e.g., patient did die or not) also the time to the endpoint is considered in the regression analysis. The hazard is modeled as: H(t)=H₀(t)·exp(w₁·V₁+w₂·V₂+w₃·V₃+ . . . ), where V₁, V₂, V₃ . . . are predictor variables and H₀(t) is the baseline hazard while H(t) is the hazard at any time t. The hazard ratio (or the risk to reach the event) is represented by Ln[H(t)/H₀(t)]=w₁·V₁+w₂·V₂+w₃·V₃+ . . . , where the coefficients or weights w₁, w₂, w₃ . . . are estimated by the Cox regression analysis and can be interpreted in a similar manner as for logistic regression analysis.

In one particular realization, the combination of the first gene expression profiles for the two or more, for example, 2, 3, 4, 5, 6, 7, 8, 9, 10, 11, 12, 13 or all, of the immune defense response genes with a regression function is determined as follows:
IDR_model:

(w₁·AIM2)+(w₂·APOBEC3A)+(w₃·CIAO1)+(w₄·DDX58)+(w₅·DHX9)+(w₆·IF116)+(w₇·IFIH1)+(w₈·IFIT1)+(w₉·IFIT3)+(w₁₀·LRRFIP1)+(w₁·MYD88)+(w₁₂·OAS1)+(w₁₃·TLR8)+(w₁₄·ZBP1) (1)

where w₁ to w₁₄ are weights and AIM2, APOBEC3A, CIAO1, DDX58, DHX9, IF116, IFIH1, IFIT1, IFIT3, LRRFIP1, MYD88, OAS1, TLR8, and ZBP1 are the expression levels of the immune defense response genes.

In one example, w₁ may be about −0.8 to 0.2, such as −0.32, w₂ may be about 0.0 to 1.0, such as 0.4743, w₃ may be about −0.2 to 0.8, such as 0.2864, w₄ may be about −1.2 to −0.2, such as −0.6683, w₅ may be about −0.9 to 0.1, such as −0.3665, w₆ may be about −0.4 to 0.6, such as 0.1357, w₇ may be about 0.0 to 1.0, such as 0.505, w₈ may be about −0.6 to 0.4, such as −0.1024, w₉ may be about 0.2 to 1.2, such as 0.7229, w₁₀ may be about −0.3 to 0.7, such as 0.2066, w₁₁ may be about −0.6 to 0.4, such as −0.1209, w₁₂ may be about −0.8 to 0.2, such as −0.2982, w₁₃ may be about −0.4 to 0.6, such as 0.1005, and w₁₄ may be about −0.7 to 0.3, such as −0.2253.

In one particular realization, the combination of the second gene expression profiles for the two or more, for example, 2, 3, 4, 5, 6, 7, 8, 9, 10, 11, 12, 13, 14, 15, 16 or all, of the T-Cell receptor signaling genes with a regression function is determined as follows:
TCR_SIGNALING_model:

(w₁₅·C2)+(w₁₆·CD247)+(w₁₇·CD28)+(w₁₈·CD3E)+(w₁₉·CD3G)+(w₂₀·CD4)+(w₂₁·CSK)+(w₂₂·EZR)+(w₂₃·FYN)+(w₂₄·LAT)+(w₂₅·LCK)+(w₂₆·PAG1)+(w₂₇·PDE4D)+(w₂₈·PRKACA)+(w₂₉·PRKACB)+(w₃₀·PTPRC)+(w₃₁·ZAP70) (2)

where w₁₅ to w₃₁ are weights and CD2, CD247, CD28, CD3E, CD3G, CD4, CSK, EZR, FYN, LAT, LCK, PAG1, PDE4D, PRKACA, PRKACB, PTPRC, and ZAP70 are the expression levels of the T-Cell receptor signaling genes.

In one example, w₁₅ may be about −1.2 to −0.2, such as −0.72, w₁₆ may be about 0.0 to 1.0, such as 0.5222, w₁₇ may be about −0.1 to 0.9, such as 0.4222, w₁₈ may be about 0.1 to 1.1, such as 0.5981, w₁₉ may be about −0.9 to 0.1, such as −0.3978, w₂₀ may be about 0.0 to 1.0, such as 0.5332, w₂₁ may be about −0.5 to 0.5, such as 0.007001, w₂₂ may be about −0.3 to 0.7, such as 0.1881, w₂₃ may be about −0.5 to 0.5, such as 0.08063, w₂₄ may be about −0.7 to 0.3, such as −0.2047, w₂₅ may be about −0.4 to 0.6, such as 0.1408, w₂₆ may be about −0.4 to 0.6, such as 0.1038, w₂₇ may be about −0.6 to 0.4, such as −0.1477, w₂₈ may be about −0.3 to 0.7, such as 0.2311, w₂₉ may be about −1.0 to 0.0, such as −0.4637, w₃₀ may be about −1.4 to −0.4, such as −0.8881, and w₃₁ may be about −0.3 to 0.7, such as 0.1936.

In one particular realization, the combination of the third gene expression profiles for the two or more, for example, 2, 3, 4, 5, 6, 7 or all, of the PDE4D7 correlated genes with a regression function is determined as follows:
PDE4D7_CORR_model:

(w₃₂·ABCC5)+(w₃₃·CUX2)+(w₃₄·KIAA1549)+(w₃₅·PDE4D)+(w₃₆·RAP1GAP2)+(w₃₇·SLC39A11)+(w₃₈·TDRD1)+(w₃₉·VWA2) (3)

where w₃₂ to w₃₉ are weights and ABCC5, CUX2, KIAA1549, PDE4D, RAP1GAP2, SLC39A11, TDRD1, and VWA2 are the expression levels of the PDE4D7 correlated genes.

In one example, w₃₂ may be about −0.4 to 0.6, such as 0.09275, w₃₃ may be about −0.2 to 0.7, such as 0.2824, w₃₄ may be about −0.8 to 0.2, such as −0.2594, w₃₅ may be about −0.5 to 0.5, such as −0.0217, w₃₆ may be about −0.4 to 0.6, such as 0.08958, w₃₇ may be about −0.7 to 0.3, such as −0.152, w₃₈ may be about −0.8 to 0.2, such as −0.2854, and w₃₉ may be about −0.6 to 0.4, such as −0.1182.

It is further preferred that the determining of the prediction of the outcome further comprises combining the combination of the first gene expression profiles, the combination of the second gene expression profiles, and the combination of the third gene expression profiles with a regression function that had been derived from a population of colorectal cancer subjects.

In one particular realization, the prediction of the outcome is determined as follows:
CRCAI_model:

(w₄₀·IDR_model)+(w₄₁·TCR_SIGNALING_model)+(w₄₂·PDE4D7_CORR_model) (4)

where w₄₀ to w₄₂ are weights, IDR_model is the above-described regression model based on the expression profiles for the two or more, for example, 2, 3, 4, 6, 7, 8, 9, 10, 11, 12, 13 or all, of the immune defense response genes, TCR_SIGNALING_model is the above-described regression model based on the expression profiles for the two or more, for example, 2, 3, 4, 6, 7, 8, 9, 10, 11, 12, 13, 14, 15, 16 or all, of the T-Cell receptor signaling genes, and PDE4D7_CORR_model is the above-described regression model based on the expression profiles for the two or more, for example, 2, 3, 4, 6, 7 or all, of the PDE4D7 correlated genes.

In one example, w₄₀ may be about 0.3 to 1.3, such as 0.7878, w₄₁ may be about 0.3 to 1.3, such as 0.7699, and w₄₂ may be about 0.1 to 1.1, such as 0.6176.

In one particular realization, the combination of the expression levels of the six or more genes are calculated as follows:
CRCAI_6 model:

(wₐ·[geneA])+(w_{b}·[geneB])+(w_{c}·[geneC])+(w_{d}·[geneD])+(wₑ·[geneE])+(w_{f}·[geneF])

where wₓ represents the weight for the respective gene geneX. Exemplary values for weights and gene combinations are listed below in Tables 3-12, however it is within the ability of the skilled artisan to generate other combinations of six or more genes as defined herein and calculated the respective weights for such model.

The prediction of the outcome may also be classified or categorized into one of at least two risk groups, based on the value of the prediction of the outcome. For example, there may be two risk groups, or three risk groups, or four risk groups, or more than four predefined risk groups. Each risk group covers a respective range of (non-overlapping) values of the prediction of the outcome. For example, a risk group may indicate a probability of occurrence of a specific clinical event from 0 to <0.1 or from 0.1 to <0.25 or from 0.25 to <0.5 or from 0.5 to 1.0 or the like.

It is further preferred that the determining of the prediction of the outcome is further based on one or more clinical parameters obtained from the subject.

As mentioned above, various measures based on clinical parameters have been investigated. By further basing the prediction of the outcome on such clinical parameter(s), it can be possible to further improve the prediction.

It is preferred that the clinical parameters comprise one or more of: (i) T stage attribute (T1, T2, T3, or T4); (ii) N stage attribute (NO, N1, or N2), and; (iii) M stage attribute (M0, M1). Additionally or alternatively, the clinical parameters comprise one or more other clinical parameters that is/are relevant for the diagnosis and/or prognosis of colorectal cancer.

It is further preferred that the determining of the prediction of the outcome comprises combining one or more of: (i) the first gene expression profile(s) for the one or more immune defense response genes; (ii) the second gene expression profile(s) for the one or more T-Cell receptor signaling genes; (iii) the third gene expression profile(s) for the one or more PDE4D7 correlated genes, and; (iv) the combination of the first gene expression profiles, the combination of the second gene expression profiles, and the combination of the third gene expression profiles, and the one or more clinical parameters obtained from the subject with a regression function that had been derived from a population of colorectal cancer subjects.

In one particular realization, the prediction of the outcome is determined as follows:
CRCAI & Clinical_Model:

(w₄₀·IDR_model)+(w₄₁·TCR_SIGNALING_model)+(w₄₂·PDE4D7_CORR_model)+(w₄₃·N_stage_N1)+(w₄₄·N_stage_N2) (5)

where w₄₀ to w₄₄ are weights, IDR_model is the above-described regression model based on the expression profiles for the two or more, for example, 2, 3, 4, 6, 7, 8, 9, 10, 11, 12, 13 or all, of the immune defense response genes, TCR_SIGNALING_model is the above-described regression model based on the expression profiles for the two or more, for example, 2, 3, 4, 6, 7, 8, 9, 10, 11, 12, 13, 14, 15, 16 or all, of the T-Cell receptor signaling genes, and PDE4D7_CORR_model is the above-described regression model based on the expression profiles for the two or more, for example, 2, 3, 4, 6, 7 or all, of the PDE4D7 correlated genes, and N_stage_N1 and N_stage_N2 are clinical N stage attributes according to the TNM classification of malignant tumors.

In one example, w₄₀ may be about 0.3 to 1.3, such as 0.8098, w₄₁ may be about 0.2 to 1.2, such as 0.7297, w₄₂ may be about 0.1 to 1.1, such as 0.6221, w₄₃ may be about 0.0 to 1.0, such as 0.4875, and w₄₄ may be about 0.4 to 1.4, such as 0.9216.

It is preferred that the biological sample is obtained from the subject before the start of the therapy. The gene expression profile(s) may be determined in the form of mRNA or protein in tissue of colorectal cancer. Alternatively, if the genes are present in a soluble form, the gene expression profile(s) may be determined in blood.

It is further preferred that the therapy is surgery, radiotherapy, cytotoxic chemotherapy (CTX), short- or long-course chemo-radiation therapy (CRT), immunotherapy, or any combination thereof.

It is preferred that the prediction of the therapy response is unlikely or likely for the effectiveness of the therapy, wherein a therapy is recommended based on the prediction and, if the prediction is negative, the recommended therapy comprises one or more of: (i) therapy provided earlier than is the standard; (ii) radiotherapy with an increased effective dose; (iii) an adjuvant therapy, such as chemotherapy; (iv) long-course CRT (chemo-radiation therapy), and; (iv) an alternative therapy, such as immunotherapy.

In a further aspect of the present invention, an apparatus for predicting an outcome of a colorectal cancer subject is presented, comprising:
an input adapted to receive data indicative of a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IF116, IFIH1, IFIT1, IFIT3, LRRFIP1, MYD88, OAS1, TLR8, and ZBP1, said first gene expression profile(s) being determined in a biological sample obtained from the subject, and/or of a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, said second gene expression profile(s) being determined in a biological sample obtained from the subject, and/or of a third gene expression profile for each of one or more, for example, 1, 2, 3, 4, 5, 6, 7 or all, PDE4D7 correlated genes selected from the group consisting of: ABCC5, CUX2, KIAA1549, PDE4D, RAP1GAP2, SLC39A11, TDRD1, and VWA2, said third gene expression profile(s) being determined in a biological sample obtained from the subject,
a processor adapted to determine the prediction of the outcome based on the first gene expression profile(s), or on the second gene expression profile(s), or on the third gene expression profile(s), or on the first, second, and third gene expression profile(s), and
optionally, a providing unit adapted to provide the prediction to a medical caregiver or the subject.

In a further aspect of the present invention, a computer program product is presented comprising instructions which, when the program is executed by a computer, cause the computer to carry out a method comprising:
receiving data indicative of a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, said first gene expression profile(s) being determined in a biological sample obtained from a colorectal cancer subject, and/or of a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, said second gene expression profile(s) being determined in a biological sample obtained from a colorectal cancer subject, and/or of a third gene expression profile for each of one or more, for example, 1, 2, 3, 4, 5, 6, 7 or all, PDE4D7 correlated genes selected from the group consisting of: ABCC5, CUX2, KIAA1549, PDE4D, RAP1GAP2, SLC39A11, TDRD1, and VWA2, said third gene expression profile(s) being determined in a biological sample obtained from a colorectal cancer subject,
determining a prediction of an outcome of the subject based on the first gene expression profile(s), or on the second gene expression profile(s), or on the third gene expression profile(s), or on the first, second, and third gene expression profile(s), and
optionally, providing the prediction to a medical caregiver or the subject.

In a further aspect of the present invention, a diagnostic kit is presented comprising:
at least one primer and/or probe for determining in a biological sample obtained from a subject, six or more gene expression levels selected from a first gene expression profile, a second gene expression profile and a third gene expression profile, wherein said six or more gene expression levels comprise at least one or more gene expression level selected from the third gene expression profile and at least one or more gene expression level selected from the first and/or the second gene expression profile, wherein
the first gene expression profile consists of the immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1;
the second gene expression profile consists of the T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70; and
the third gene expression profile consisting of the PDE4D7 correlated genes selected from the group consisting of: ABCC5, CUX2, KIAA1549, PDE4D, RAP1GAP2, SLC39A11, TDRD1, and VWA2; and
optionally, an apparatus as defined in claim 11 or a computer program product as defined in claim 12.

Alternatively a diagnostic kit is disclosed comprising:
at least one primer and/or probe for determining a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, in a biological sample obtained from a subject, and/or
at least one primer and/or probe for determining a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, in a biological sample obtained from a subject, and/or
at least one primer and/or probe for determining a gene expression profile for each of one or more, for example, 1, 2, 3, 4, 5, 6, 7 or all, PDE4D7 correlated genes selected from the group consisting of: ABCC5, CUX2, KIAA1549, PDE4D, RAP1GAP2, SLC39A11, TDRD1, and VWA2, in a biological sample obtained from a subject, and
optionally, an apparatus as defined in claim 11 or a computer program product as defined in claim 12.

In a further aspect of the present invention, a use of the kit as defined in claim 13 is presented.

It is preferred that the use as defined in claim 14 is in a method of predicting an outcome of a colorectal cancer subject.

In a further aspect of the present invention, a method is presented, comprising:
receiving one or more biological sample(s) obtained from a colorectal cancer subject,
using the kit as defined in claim 13 to determine six or more gene expression levels selected from a first gene expression profile, a second gene expression profile and a third gene expression profile, wherein said six or more gene expression levels comprise at least one or more gene expression level selected from the third gene expression profile and at least one or more gene expression level selected from the first and/or the second gene expression profile, wherein
the first gene expression profile consists of the immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1;
the second gene expression profile consists of the T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70; and
the third gene expression profile consists of the PDE4D7 correlated genes selected from the group consisting of: ABCC5, CUX2, KIAA1549, PDE4D, RAP1GAP2, SLC39A11, TDRD1, and VWA2.

Alternatively, a method is presented comprising:
receiving one or more biological sample(s) obtained from a colorectal cancer subject,
using the kit as defined in claim 13 to determine a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, in a biological sample obtained from the subject, and/or
using the kit as defined in claim 13 to determine a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, in a biological sample obtained from the subject, and/or
using the kit as defined in claim 13 to determine a third gene expression profile for each of one or more, for example, 1, 2, 3, 4, 5, 6, 7 or all, PDE4D7 correlated genes selected from the group consisting of: ABCC5, CUX2, KIAA1549, PDE4D, RAP1GAP2, SLC39A11, TDRD1, and VWA2, in a biological sample obtained from the subject.

In a further aspect of the present invention, a use of six or more gene expression levels selected from a first gene expression profile, a second gene expression profile and a third gene expression profile, wherein said six or more gene expression levels comprise at least one or more gene expression level selected from the third gene expression profile and at least one or more gene expression level selected from the first and/or the second gene expression profile, wherein the first gene expression profile consists of the immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, the second gene expression profile consists of the T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70 the third gene expression profile consists of the PDE4D7 correlated genes selected from the group consisting of: ABCC5, CUX2, KIAA1549, PDE4D, RAP1GAP2, SLC39A11, TDRD1, and VWA2, in a method of predicting an outcome of a colorectal cancer subject, comprising:
determining the prediction of the outcome based on the six or more gene expression levels, and
optionally, providing the prediction to a medical caregiver or the subject.

Alternatively the method relates to the use of a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, and/or of a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, and/or of a third gene expression profile for each of one or more, for example, 1, 2, 3, 4, 5, 6, 7 or all, PDE4D7 correlated genes selected from the group consisting of: ABCC5, CUX2, KIAA1549, PDE4D, RAP1GAP2, SLC39A11, TDRD1, and VWA2, in a method of predicting an outcome of a colorectal cancer subject is presented, comprising:
determining the prediction of the outcome based on the first gene expression profile(s), or on the second gene expression profile(s), or on the third gene expression profile(s), or on the first, second, and third gene expression profile(s), and
optionally, providing the prediction or the personalization or a therapy recommendation based on the prediction or the personalization to a medical caregiver or the subject.

It shall be understood that the method of claim 1, the apparatus of claim 11, the computer program product of claim 12, the diagnostic kit of claim 13, the use of the diagnostic kit of claim 14, the method of claim 16, and the use of first, second, and/or third gene expression profile(s) of claim 17 have similar and/or identical preferred embodiments, in particular, as defined in the dependent claims.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the Following Drawings:

FIG. 1 shows schematically and exemplarily a flowchart of an embodiment of a method of predicting an outcome of a colorectal cancer subject,

FIG. 2 shows a Kaplan-Meier curve of the IDR_model in a 168 patient TCGA colorectal cancer cohort (training set used to develop the IDR_model),

FIG. 3 shows a Kaplan-Meier curve of the IDR_model in a 83 patient TCGA colorectal cancer cohort (testing set used to validate the IDR_model as developed on the 168 patient training set),

FIG. 4 shows a Kaplan-Meier curve of the TCR_SIGNALING_model in a 168 patient TCGA colorectal cancer cohort (training set used to develop the TCR_SIGNALING_model),

FIG. 5 shows a Kaplan-Meier curve of the TCR_SIGNALING_model in a 83 patient TCGA colorectal cancer cohort (testing set used to validate the TCR_SIGNALING_model as developed on the 168 patient training set),

FIG. 6 shows a Kaplan-Meier curve of the PDE4D7_CORR_model in a 168 patient TCGA colorectal cancer cohort (training set used to develop the PDE4D7_CORR_model),

FIG. 7 shows a Kaplan-Meier curve of the PDE4D7_CORR_model in a 83 patient TCGA colorectal cancer cohort (testing set used to validate the PDE4D7_CORR_model as developed on the 168 patient training set),

FIG. 8 shows a Kaplan-Meier curve of the CRCAI_model in a 168 patient TCGA colorectal cancer cohort (training set used to develop the CRCAI_model),

FIG. 9 shows a Kaplan-Meier curve of the CRCAI_model in a 83 patient TCGA colorectal cancer cohort (testing set used to validate the CRCAI_model as developed on the 168 patient training set),

FIG. 10 shows a Kaplan-Meier curve of the CRCAI & Clinical_model in a 168 patient TCGA colorectal cancer cohort (training set used to develop the CRCAI & Clinical_model),

FIG. 11 shows a Kaplan-Meier curve of the CRCAI & Clinical_model in a 83 patient TCGA colorectal cancer cohort (testing set used to validate the CRCAI & Clinical_model as developed on the 168 patient training set),

FIG. 12 shows a Kaplan-Meier curve of the CRCAI_model in a colorectal cancer cohort derived from the GSE41248 data set. The clinical endpoint that was tested was the overall death,

FIG. 13 shows a Kaplan-Meier curve of the CRCAI_model in a colorectal cancer cohort derived from the GSE41248 data set. The clinical endpoint that was tested was the cancer specific death,

FIG. 14 shows a Kaplan-Meier curve of the CRCAI & Clinical_model in a colorectal cancer cohort derived from the GSE41248 data set. The clinical endpoint that was tested was the overall death, and

FIG. 15 shows a Kaplan-Meier curve of the CRCAI & Clinical_model in a colorectal cancer cohort derived from the GSE41248 data set. The clinical endpoint that was tested was the cancer specific death.

### DETAILED DESCRIPTION OF EMBODIMENTS

### Overview Of Outcome Prediction

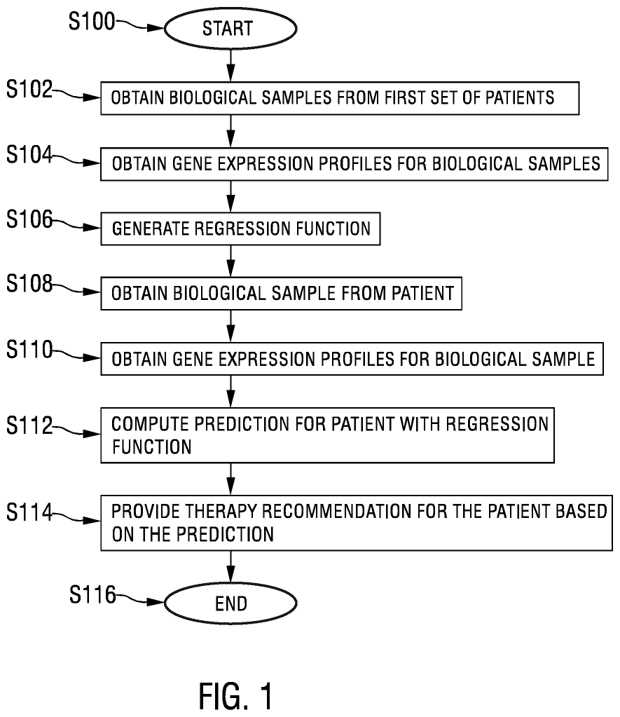

FIG. 1 shows schematically and exemplarily a flowchart of an embodiment of a method of predicting an outcome of a colorectal cancer subject.

The method begins at step S100.

At step S102, a biological sample is obtained from each of a first set of patients (subjects) diagnosed with colorectal cancer. Preferably, monitoring colorectal cancer has been performed for these colorectal cancer patients over a period of time, such as at least one year, or at least two years, or about five years, after obtaining the biological sample.

At step S104, a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, and/or a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, and/or a third gene expression profile for each of two or more, for example, 2, 3, 4, 5, 6, 7 or all, PDE4D7 correlated genes selected from the group consisting of: ABCC5, CUX2, KIAA1549, PDE4D, RAP1GAP2, SLC39A11, TDRD1, and VWA2, is obtained for each of the biological samples obtained from the first set of patients, e.g., by performing RT-qPCR (real-time quantitative PCR) on RNA extracted from each biological sample. The exemplary gene expression profiles include an expression level (e.g., value) for each of the two or more genes which can be normalized using value(s) for each of a set of reference genes, such as B2M, HPRT1, POLR2A, and/or PUM1. In one realization, the gene expression level for each of the two or more genes of the first gene expression profiles, and/or the second gene expression profiles, and/or the third gene expression profiles is normalized with respect to one or more reference genes selected from the group consisting of ACTB, ALAS1, B2M, HPRT1, POLR2A, PUM1, RPLP0, TBP, TUBA1B, and/or YWHAZ, e.g., at least one, or at least two, or at least three, or, preferably, all of these reference genes.

At step S106, a regression function for assigning a prediction of the outcome is determined based on the first gene expression profiles for the two or more immune defense response genes, AIM2, APOBEC3A, CIAO1, DDX58, DHX9, IFI16, IFIH1, IFIT1, IFIT3, LRRFIP1, MYD88, OAS1, TLR8, and/or ZBP1, and/or the second gene expression profiles for the two or more T-Cell receptor signaling genes, CD2, CD247, CD28, CD3E, CD3G, CD4, CSK, EZR, FYN, LAT, LCK, PAG1, PDE4D, PRKACA, PRKACB, PTPRC, and/or ZAP70, and/or the third gene expression profiles for the two or more PDE4D7 correlated genes, ABCC5, CUX2, KIAA1549, PDE4D, RAP1GAP2, SLC39A11, TDRD1, and/or VWA2, obtained for at least some of the biological samples obtained for the first set of patients and respective results obtained from the monitoring. In one particular realization, the regression function is determined as specified in Eq. (4) above.

At step S108, a biological sample is obtained from a patient (subject or individual). The patient can be a new patient or one of the first set.

At step S110, a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, and/or a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, and/or a third gene expression profile is obtained for each of the two or more, for example, 2, 3, 4, 5, 6, 7 or all, PDE4D7 correlated genes, e.g., by performing PCR on the biological sample. In one realization, the gene expression level for each of the two or more genes of the first gene expression profiles, and/or the second gene expression profiles, and/or the third gene expression profiles is normalized with respect to one or more reference genes selected from the group consisting of ACTB, ALAS1, B2M, HPRT1, POLR2A, PUM1, RPLP0, TBP, TUBA1B, and/or YWHAZ, e.g., at least one, or at least two, or at least three, or, preferably, all of these reference genes. This is substantially the same as in step S104.

At step S112, a prediction of the outcome based on the first, second, and third gene expression profiles is determined for the patient using the regression function. This will be described in more detail later in the description.

At S114, a therapy recommendation may be provided, e.g., to the patient or his or her guardian, to a doctor, or to another healthcare worker, based on the prediction or the personalization. To this end, the prediction or personalization may be categorized into one of a predefined set of risk groups, based on the value of the prediction or personalization. In one particular realization, the therapy may be radiotherapy and the prediction of the therapy response may be unlikely or likely for the effectiveness of the therapy. If the prediction is unlikely, the recommended therapy may comprise one or more of: (i) therapy provided earlier than is the standard; (ii) radiotherapy with an increased effective dose; (iii) an adjuvant therapy, such as chemotherapy; (iv) long-course CRT (chemo-radiation therapy), and; (iv) an alternative therapy, such as immunotherapy.

The method ends at S116.

In one embodiment, the gene expression profiles at steps S104 and S110 are determined by detecting mRNA expression using two or more primers and/or probes and/or two or more sets thereof.

In one embodiment, steps S104 and S110 further comprise obtaining clinical parameters from the first set of patients and the patient, respectively. The clinical parameters may comprise one or more of: (i) T stage attribute (T1, T2, T3, or T4); (ii) N stage attribute (NO, N1, or N2), and; (iii) M stage attribute (M0, M1). Additionally or alternatively, the clinical parameters comprise one or more other clinical parameters that is/are relevant for the diagnosis and/or prognosis of colorectal cancer. The regression function for assigning the prediction of the outcome that is determined in step S106 is then further based on the one or more clinical parameters obtained from at least some of the first set of patients. In step S112, the prediction of the outcome is then further based on the one or more clinical parameters, e.g., the N stage attribute, obtained from the patient and is determined for the patient using the regression function. In one particular realization, the regression function is determined as specified in Eq. (5) above.

Based on the significant correlation with survival outcome after therapy, we expect that the identified molecules will provide predictive value with regard to the effectiveness of the treatment of primary colorectal cancers.

### Results

For each gene, the log 2 expression value as provided in the download from the TCGA database (TCGA Colorectal Adenocarcinoma, Firehose legacy, http://linkedomics.org/login.php, accessed Mar. 6, 2020) was obtained.

The log 2 expression values for each gene were transformed into z-scores by calculating:

log 2_gene transformed z-score=((log 2_gene)−(mean_samples))/(stdev_samples) (6)

where log 2_gene is the log 2 gene expression value per gene, mean_samples is the mathematical mean of the log 2_gene values across all samples, and stdev_samples is the standard deviation of the log 2_gene values across all samples.

This process distributes the transformed log 2_gene values around the mean 0 with a standard deviation of 1.

For the multivariate analysis of the genes of interest we used the log 2_gene transformed z-score value of each gene as input.

### Cox Regression Analysis

We then set out to test whether the combination of the 14 immune defense response genes, the combination of the 17 T-Cell receptor signalling genes, the combination of the eight PDE4D7 correlated genes, and a combination thereof will exhibit a prognostic value for colorectal cancer. With Cox regression we modelled the expression levels of the 14 immune defense response genes, of the 17 T-Cell receptor signalling genes, and of the eight PDE4D7 correlated genes, respectively, to overall survival in a TCGA cohort of 377 colorectal cancer patients.

From the TCGA colorectal cancer cohort, only samples with combined presence of clinical parameters, gene expression values, and survival information were included. From this subset, we only included samples from patients with non-metastasized disease (m0), which resulted in a total number of 251 patients. These 251 patients were randomly split into 3 groups. Cohort 1 (n=168) was used as a train cohort and consisted of groups 1+2. Cohort 2 (n=83) consisted of group 3 and was used to validate the risk models as derived from the train cohort.

The Cox regression functions were derived as follows:

### IDR_Model:

(w₁·AIM2)+(w₂·APOBEC3A)+(w₃·CIAO1)+(w₄·DDX58)+(w₅·DHX9)+(w₆·IF116)+(w₇·IFIH1)+(w₈·IFIT1)+(w₉·IFIT3)+(w₁₀·LRRFIP1)+(w₁₁·MYD88)+(w₁₂·OAS1)+(w₁₃·TLR8)+(w₁₄·ZBP1)

TCR_SIGNALING_Model:

(w₁₅·C2)+(w₁₆·CD247)+(w₁₇·CD28)+(w₁₈·CD3E)+(w₁₉·CD3G)+(w₂₀·CD4)+(w₂₁·CSK)+(w₂₂·EZR)+(w₂₃·FYN)+(w₂₄·LAT)+(w₂₅·LCK)+(w₂₆·PAG1)+(w₂₇·PDE4D)+(w₂₈·PRKACA)+(w₂₉·PRKACB)+(w₃₀·PTPRC)+(w₃₁·ZAP70)

PDE4D7_CORR_Model:

(w₃₂·ABCC5)+(w₃₃·CUX2)+(w₃₄·KIAA1549)+(w₃₅·PDE4D)+(w₃₆·RAP1GAP2)+(w₃₇·SLC39A11)+(w₃₈·TDRD1)+(w₃₉·VWA2)

The details for the weights w₁ to w₃₉ are shown in the following TABLE 1.

TABLE 1
Variables and weights for the three individual Cox regression models,
i.e., the immune defense response model (IDR_model), the T-Cell
receptor signaling model (TCR SIGNALING model), and the PDE4D7 correlation model
(PDE4D7_CORR_model) for colorectal cancer; NA—not available.

| Variable | Weights | | | |
|---|---|---|---|---|
| Model | | IDR_model | TCR_SIGNALING_model | PDE4D7_CORR_model |
| AIM2 | w₁ | −0.32 | NA | NA |
| APOBEC3A | w₂ | 0.4743 | NA | NA |
| CIAO1 | w₃ | 0.2864 | NA | NA |
| DDX58 | w₄ | −0.6683 | NA | NA |
| DHX9 | w₅ | −0.3665 | NA | NA |
| IFI16 | w₆ | 0.1357 | NA | NA |
| IFIH1 | w₇ | 0.505 | NA | NA |
| IFIT1 | w₈ | −0.1024 | NA | NA |
| IFIT3 | w₉ | 0.7229 | NA | NA |
| LRRFIP1 | w₁₀ | 0.2066 | NA | NA |
| MYD88 | w₁₁ | −0.1209 | NA | NA |
| OAS1 | w₁₂ | −0.2982 | NA | NA |
| TLR8 | w₁₃ | 0.1005 | NA | NA |
| ZBP1 | w₁₄ | −0.2253 | NA | NA |
| CD2 | w₁₅ | NA | −0.72 | NA |
| CD247 | w₁₆ | NA | 0.5222 | NA |
| CD28 | w₁₇ | NA | 0.4222 | NA |
| CD3E | w₁₈ | NA | 0.5981 | NA |
| CD3G | w₁₉ | NA | −0.3978 | NA |
| CD4 | w₂₀ | NA | 0.5332 | NA |
| CSK | w₂₁ | NA | 0.007001 | NA |
| EZR | w₂₂ | NA | 0.1881 | NA |
| FYN | w₂₃ | NA | 0.08063 | NA |
| LAT | w₂₄ | NA | −0.2047 | NA |
| LCK | w₂₅ | NA | 0.1408 | NA |
| PAG1 | w₂₆ | NA | 0.1038 | NA |
| PDE4D | w₂₇ | NA | −0.1477 | NA |
| PRKACA | w₂₈ | NA | 0.2311 | NA |
| PRKACB | w₂₉ | NA | −0.4637 | NA |
| PTPRC | w₃₀ | NA | −0.8881 | NA |
| ZAP70 | w₃₁ | NA | 0.1936 | NA |
| ABCC5 | w₃₂ | NA | NA | 0.09275 |
| CUX2 | w₃₃ | NA | NA | 0.2824 |
| KIAA1549 | w₃₄ | NA | NA | −0.2594 |
| PDE4D | w₃₅ | NA | NA | −0.0217 |
| RAP1GAP2 | w₃₆ | NA | NA | 0.08958 |
| SLC39A11 | w₃₇ | NA | NA | −0.152 |
| TDRD1 | w₃₈ | NA | NA | −0.2854 |
| VWA2 | w₃₉ | NA | NA | −0.1182 |

Based on the three individual Cox regression models (IDR_model, TCR_SIGNALING_model, PDE4D7_CORR_model) we then again used Cox regression to model the combination thereof to overall survival with (CRCAI & Clinical_model) or without (CRCAI_model) the presence of the clinical variables (N stage attributes) in the respective cohorts of colorectal cancer patients. We tested the two models in Kaplan-Meier survival analysis.

The Cox regression functions were derived as follows:

CRCAI_Model:

(w₄₀·IDR_model)+(w₄₁·TCR_SIGNALING_model)+(w₄₂·PDE4D7_CORR_model)

CRCAI & Clinical_Model:

(w₄₀·IDR_model)+(w₄₁·TCR_SIGNALING_model)+(w₄₂·PDE4D7_CORR_model)+(w₄₃·N_stage_N1)+(w₄₄·N_stage_N2)

The details for the weights w₄₀ to w₄₄ are shown in the following TABLE 2.

TABLE 2
Variables and weights for two combination Cox regression models,
i.e., colorectal cancer AI model (CRCAI_model) and the colorectal cancer
& clinical model (CRCAI&Clinical_model); NA—not available.

| Variable | Weights | | |
|---|---|---|---|
| Model | | CRCAI_model | CRCAI&Clinical_model |
| IDR_model | w₄₀ | 0.7878 | 0.8098 |
| TCR_SIGNALING_model | w₄₁ | 0.7699 | 0.7297 |
| PDE47_CORR_model | w₄₂ | 0.6176 | 0.6221 |
| N_stage_N1 | w₄₃ | NA | 0.3293 |
| N_stage_N2 | w₄₄ | NA | 0.9216 |

### Kaplan-Meier Survival Analysis

For Kaplan-Meier survival curve analysis, the Cox functions of the risk models (IDR_model, TCR_SIGNALING_model, PDE4D7_CORR_model, CRCAI_model, and CRCAI & Clinical_model) were categorized into two sub-cohorts based on a cut-off. The threshold for group separation into low and high risk was based on the risk to experience the clinical endpoint (outcome) as predicted by the respective Cox regression model

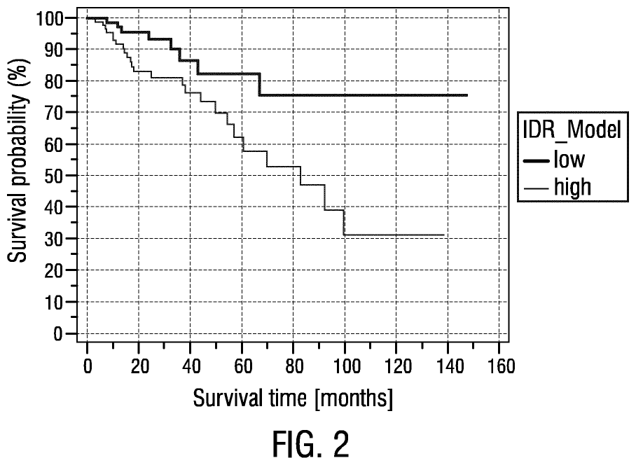

FIG. 2 shows a Kaplan-Meier curve of the IDR_model in a 168 patient TCGA colorectal cancer cohort (training set used to develop the IDR_model). The clinical endpoint that was tested was the overall death (logrank p=0.009; HR=2.5; 95% CI=1.3-5.0). The following supplementary lists indicate the number of patients at risk for the IDR_model classes analyzed (threshold=0), i.e., the patients at risk at any time interval+20 months are shown: Low risk: 80, 45, 21, 15, 9, 6, 4, 1, 0; High risk: 88, 52, 29, 14, 9, 4, 3, 0, 0.

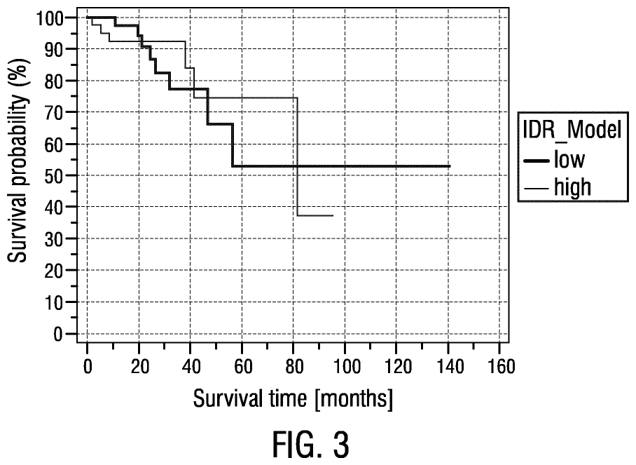

FIG. 3 shows a Kaplan-Meier curve of the IDR_model in a 83 patient TCGA colorectal cancer cohort (testing set used to validate the IDR_model as developed on the 168 patient training set). The clinical endpoint that was tested was the overall death (logrank p=0.8; HR=0.9; 95% CI=0.3-2.6). The following supplementary lists indicate the number of patients at risk for the IDR_model classes analyzed (threshold=0), i.e., the patients at risk at any time interval+20 months are shown: Low risk: 42, 29, 8, 4, 4, 2, 2, 1, 0; High risk: 41, 19, 9, 4, 2, 0, 0, 0, 0.

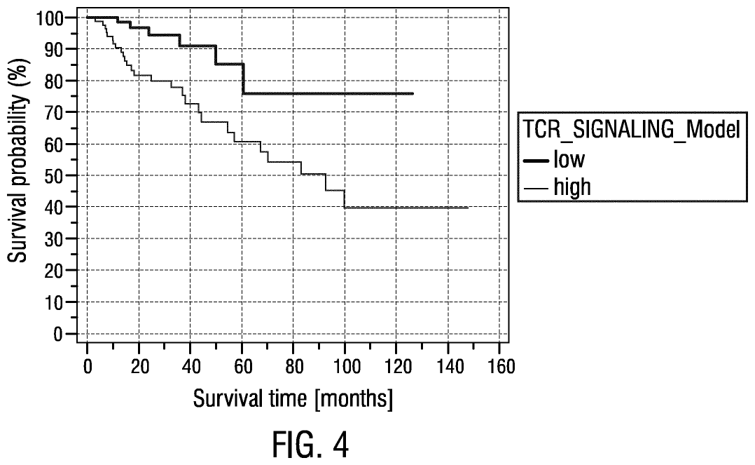

FIG. 4 shows a Kaplan-Meier curve of the TCR_SIGNALING_model in a 168 patient TCGA colorectal cancer cohort (training set used to develop the TCR_SIGNALING_model). The clinical endpoint that was tested was the overall death (logrank p=0.003; HR=2.9; 95% CI=1.4-5.9). The following supplementary lists indicate the number of patients at risk for the TCR_SIGNALING_model classes analyzed (threshold=0), i.e., the patients at risk at any time interval +20 months are shown: Low risk: 81, 46, 23, 10, 4, 3, 1, 0, 0; High risk: 87, 51, 27, 19, 14, 7, 6, 1, 0.

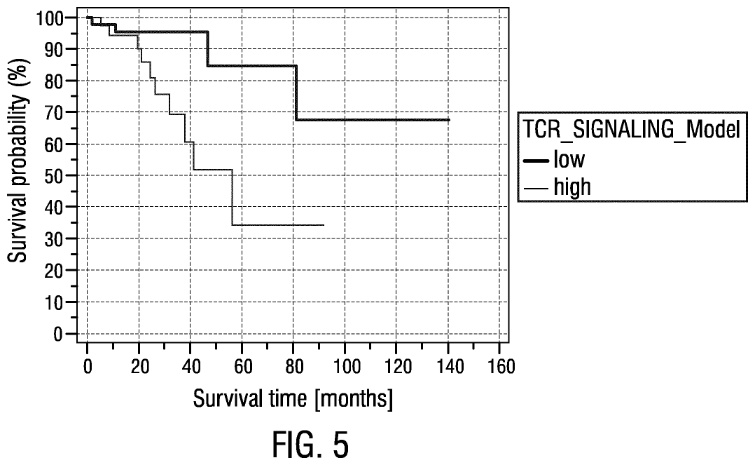

FIG. 5 shows a Kaplan-Meier curve of the TCR_SIGNALING_model in a 83 patient TCGA colorectal cancer cohort (testing set used to validate the TCR_SIGNALING_model as developed on the 168 patient training set). The clinical endpoint that was tested was the overall death (logrank p=0.02; HR=3.7; 95% CI=1.3-10.9). The following supplementary lists indicate the number of patients at risk for the TCR_SIGNALING_model classes analyzed (threshold=0), i.e., the patients at risk at any time interval+20 months are shown: Low risk: 46, 26, 10, 6, 5, 2, 2, 1, 0; High risk: 37, 22, 7, 2, 1, 0, 0, 0, 0.

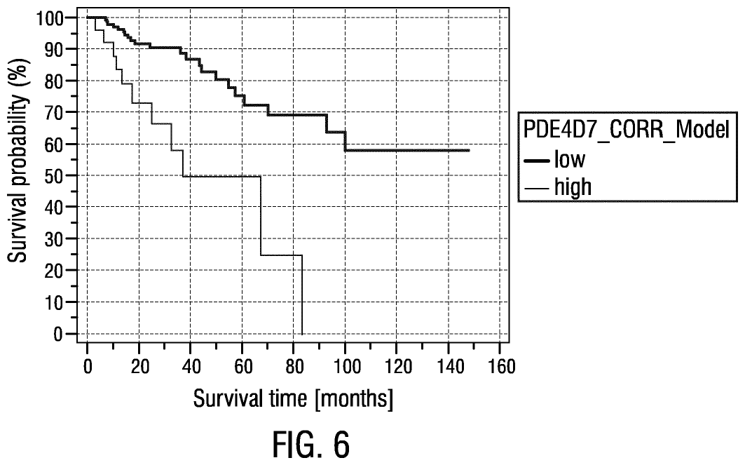

FIG. 6 shows a Kaplan-Meier curve of the PDE4D7_CORR_model in a 95 patient TCGA colorectal cancer cohort (training set used to develop the PDE4D7_CORR_model). The clinical endpoint that was tested was the overall death (logrank p<0.0001; HR=10.7; 95% CI=3.5-32.4). The following supplementary lists indicate the number of patients at risk for the PDE4D7_CORR_model classes analyzed (threshold=0), i.e., the patients at risk at any time interval +20 months are shown: Low risk: 143, 86, 45, 27, 17, 10, 7, 1, 0; High risk: 25, 11, 5, 2, 1, 0, 0, 0, 0.

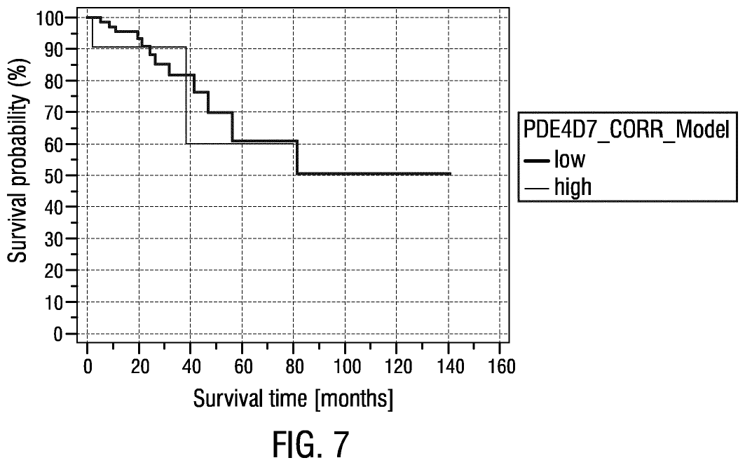

FIG. 7 shows a Kaplan-Meier curve of the PDE4D7_CORR_model in a 83 patient TCGA colorectal cancer cohort (testing set used to validate the PDE4D7_CORR_model as developed on the 168 patient training set). The clinical endpoint that was tested was the overall death (logrank p<0.8; HR=1.2; 95% CI=0.2-5.9). The following supplementary lists indicate the number of patients at risk for the PDE4D7_CORR_model classes analyzed (threshold=0), i.e., the patients at risk at any time interval+20 months are shown: Low risk: 72, 42, 15, 7, 6, 2, 2, 1, 0; High risk: 11, 6, 2, 1, 0, 0, 0, 0, 0.

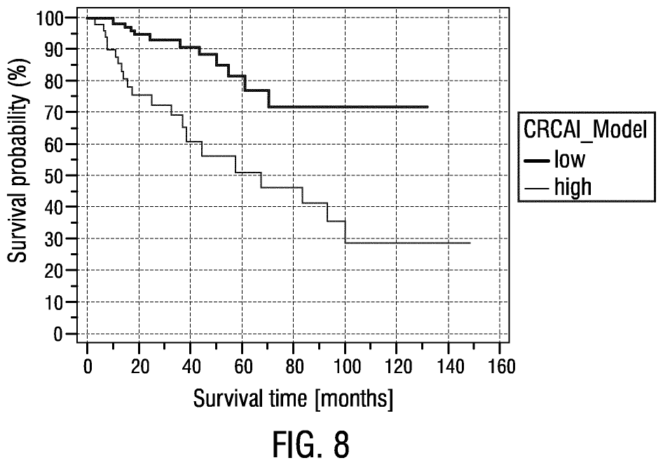

FIG. 8 shows a Kaplan-Meier curve of the CRCAI_model in a 168 patient TCGA colorectal cancer cohort (training set used to develop the CRCAI_model). The clinical endpoint that was tested was the overall death (logrank p=0.0001; HR=4.6; 95% CI=2.2-9.7). The following supplementary lists indicate the number of patients at risk for the CRCAI_model classes analyzed (threshold=0.5), i.e., the patients at risk at any time interval+20 months are shown: Low risk: 117, 69, 37, 19, 9, 6, 3, 0, 0; High risk: 51, 28, 13, 10, 9, 4, 4, 1, 0.

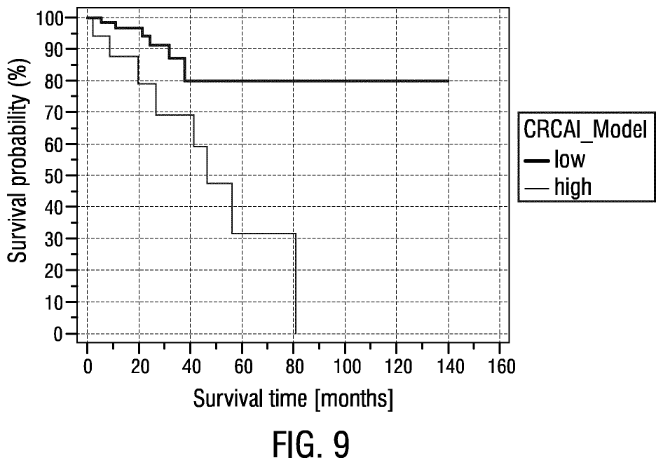

FIG. 9 shows a Kaplan-Meier curve of the CRCAI_model in a 83 patient TCGA colorectal cancer cohort (testing set used to validate the CRCAI_model as developed on the 168 patient training set). The clinical endpoint that was tested was the overall death (logrank p=0.005; HR=5.9; 95% CI=1.7-20.2). The following supplementary lists indicate the number of patients at risk for the CRCAI_model classes analyzed (threshold=0.5), i.e., the patients at risk at any time interval +20 months are shown: Low risk: 66, 39, 10, 6, 5, 2, 2, 1, 0; High risk: 17, 9, 7, 2, 1, 0, 0, 0, 0.

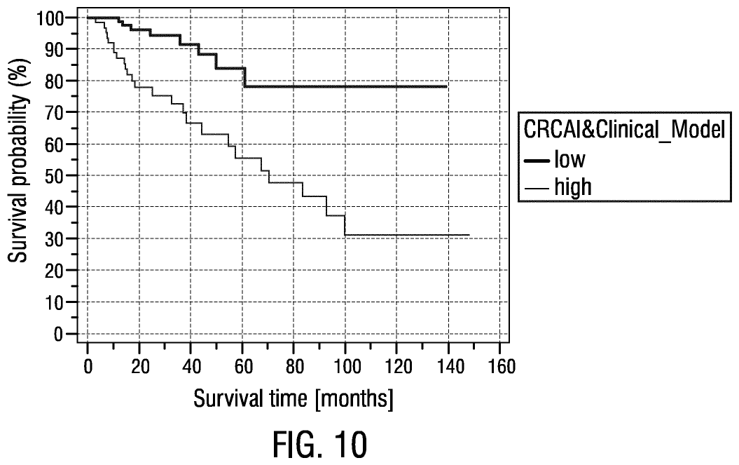

FIG. 10 shows a Kaplan-Meier curve of the CRCAI & Clinical_model in a 168 patient TCGA colorectal cancer cohort (training set used to develop the CRCAI & Clinical_model). The clinical endpoint that was tested was the overall death (logrank p=0.0001; HR=4.0; 95% CI=2.0-8.0). The following supplementary lists indicate the number of patients at risk for the CRCAI & Clinical_model classes analyzed (threshold=0.5), i.e., the patients at risk at any time interval +20 months are shown: Low risk: 105, 59, 29, 15, 7, 5, 3, 0, 0; High risk: 63, 38, 21, 14, 11, 5, 4, 1, 0.

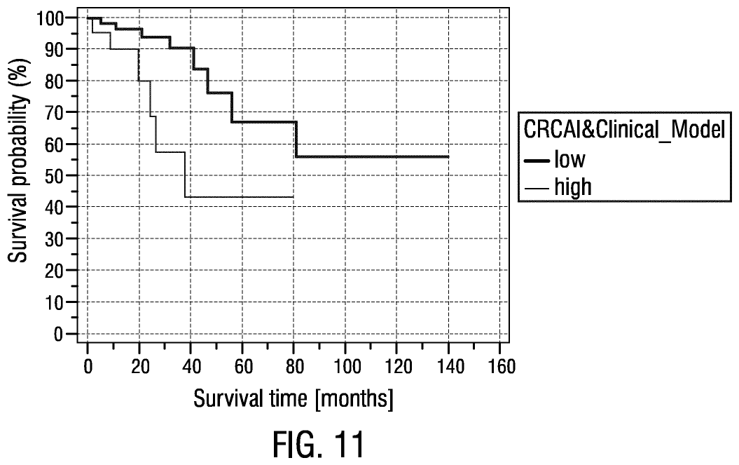

FIG. 11 shows a Kaplan-Meier curve of the CRCAI & Clinical_model in a 83 patient TCGA colorectal cancer cohort (testing set used to validate the CRCAI & Clinical_model as developed on the 168 patient training set). The clinical endpoint that was tested was the overall death (logrank p=0.01; HR=5.5; 95% CI=1.4-21.8). The following supplementary lists indicate the number of patients at risk for the CRCAI & Clinical_model classes analyzed (threshold=0.5), i.e., the patients at risk at any time interval+20 months are shown: Low risk: 62, 40, 14, 7, 6, 2, 2, 1, 0; High risk: 21, 8, 3, 1, 0, 0, 0, 0, 0.

In the following, we show some additional results for a colorectal cancer cohort derived from the GSE41248 (www.ncbi.nlm.nih.gov/geo) data set:

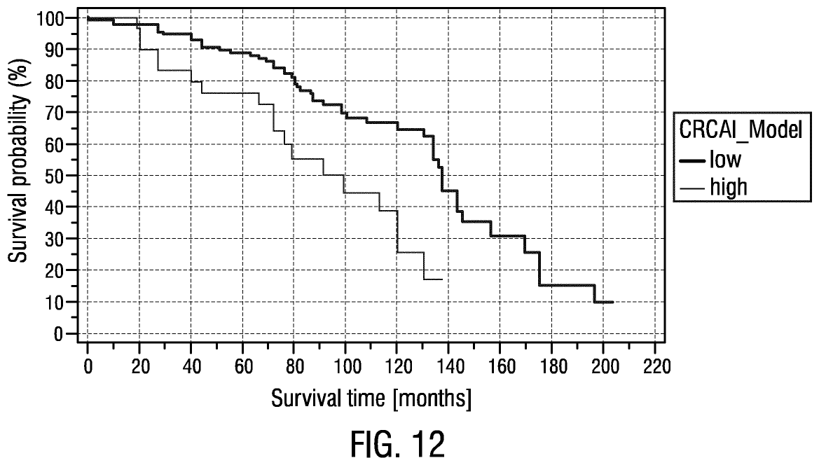

FIG. 12 shows a Kaplan-Meier curve of the CRCAI_model in a colorectal cancer cohort derived from the GSE41248 data set. The model was trained on ⅔ of the samples from patients with non-metastasized (m0) and metastasized disease (ml) and then tested on the remaining ⅓ of the m0 and ml samples. This trained model was then tested only on samples from patients with non-metastasized disease (m0) in order to have the same situation as with the TCGA colorectal cancer cohort. The clinical endpoint that was tested was the overall death (logrank p=0.001; HR=3.2; 95% CI=1.6-6.5). The following supplementary lists indicate the number of patients at risk for the CRCAI_model classes analyzed (threshold=0.5), i.e., the patients at risk at any time interval+20 months are shown: Low risk: 132, 123, 113, 97, 75, 50, 32, 14, 6, 3, 2, 0; High risk: 31, 27, 23, 22, 12, 8, 4, 0, 0, 0, 0, 0.

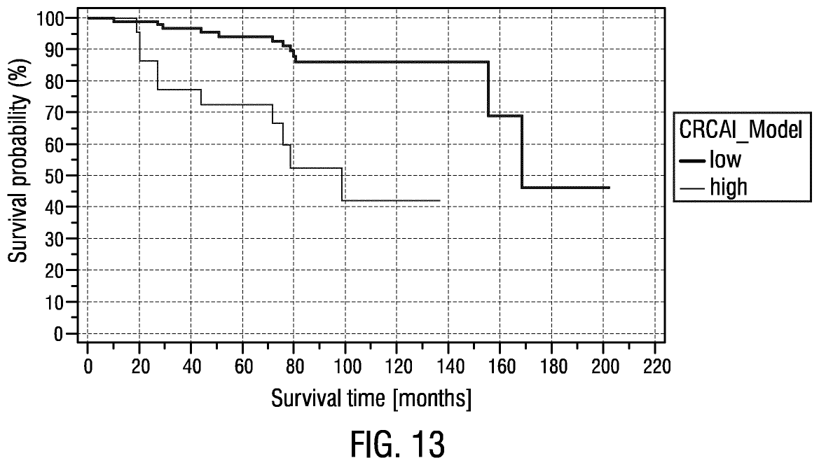

FIG. 13 shows a Kaplan-Meier curve of the CRCAI_model in a colorectal cancer cohort derived from the GSE41248 data set. The model was trained on ⅔ of the samples from patients with non-metastasized (m0) and metastasized disease (m Il) and then tested on the remaining ⅓ of the m0 and ml samples. This trained model was then tested only on samples from patients with non-metastasized disease (m0) in order to have the same situation as with the TCGA colorectal cancer cohort. The clinical endpoint that was tested was the cancer specific death (logrank p<0.0001; HR=12.9; 95% CI=3.9-43.0). The following supplementary lists indicate the number of patients at risk for the CRCAI_model classes analyzed (threshold=0.5), i.e., the patients at risk at any time interval+20 months are shown: Low risk: 94, 86, 80, 67, 51, 34, 18, 8, 3, 2, 2, 0; High risk: 23, 19, 16, 15, 7, 4, 3, 0, 0, 0, 0, 0.

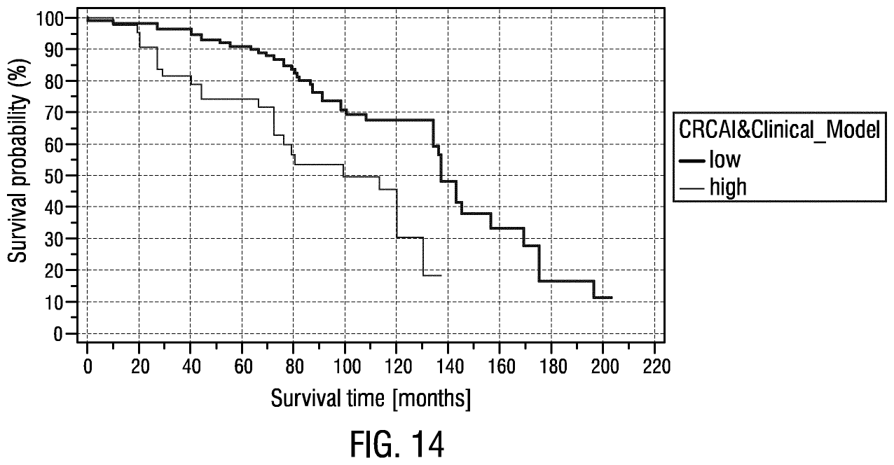

FIG. 14 shows a Kaplan-Meier curve of the CRCAI & Clinical_model in a colorectal cancer cohort derived from the GSE41248 data set. The model was trained on ⅔ of the samples from patients with non-metastasized (m0) and metastasized disease (ml) and then tested on the remaining ⅓ of the m0 and ml samples. This trained model was then tested only on samples from patients with non-metastasized disease (m0) in order to have the same situation as with the TCGA colorectal cancer cohort. The clinical endpoint that was tested was the overall death (logrank p=0.0002; HR=3.3; 95% CI=1.8-6.1). The following supplementary lists indicate the number of patients at risk for the CRCAI & Clinical_model classes analyzed (threshold=0.5), i.e., the patients at risk at any time interval +20 months are shown: Low risk: 120, 111, 103, 90, 71, 45, 30, 14, 6, 3, 2, 0; High risk: 43, 39, 33, 29, 16, 13, 6, 0, 0, 0, 0, 0.

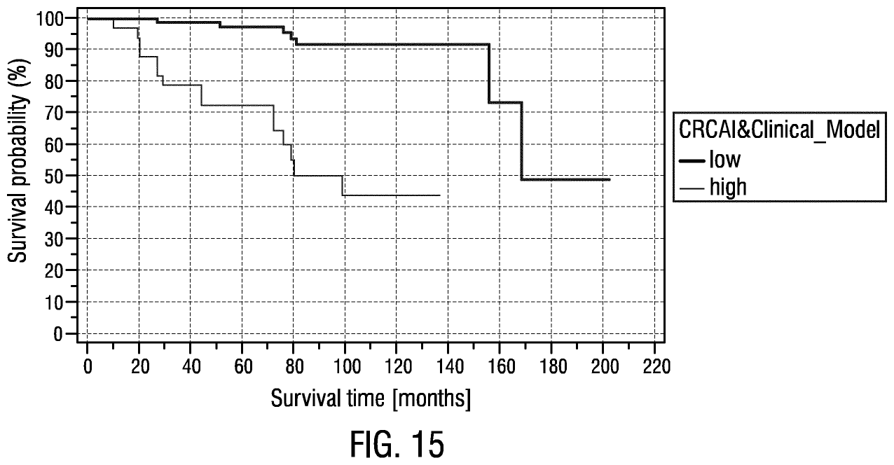

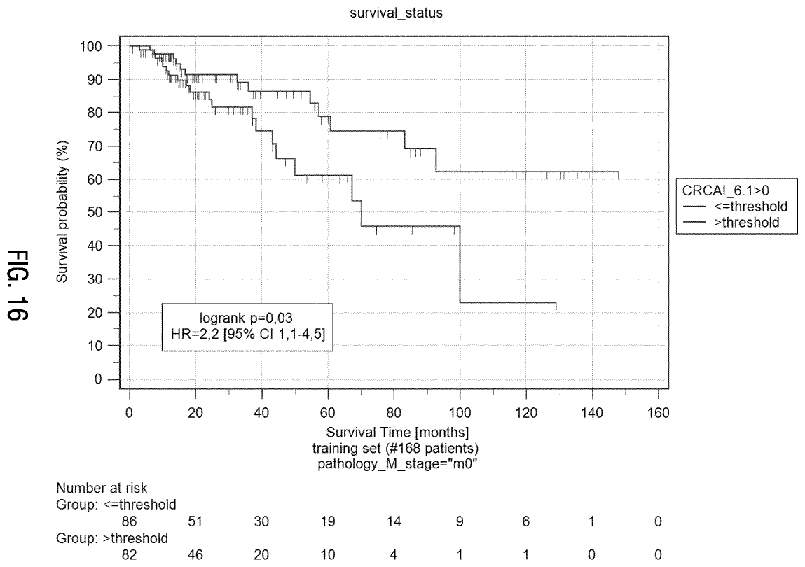

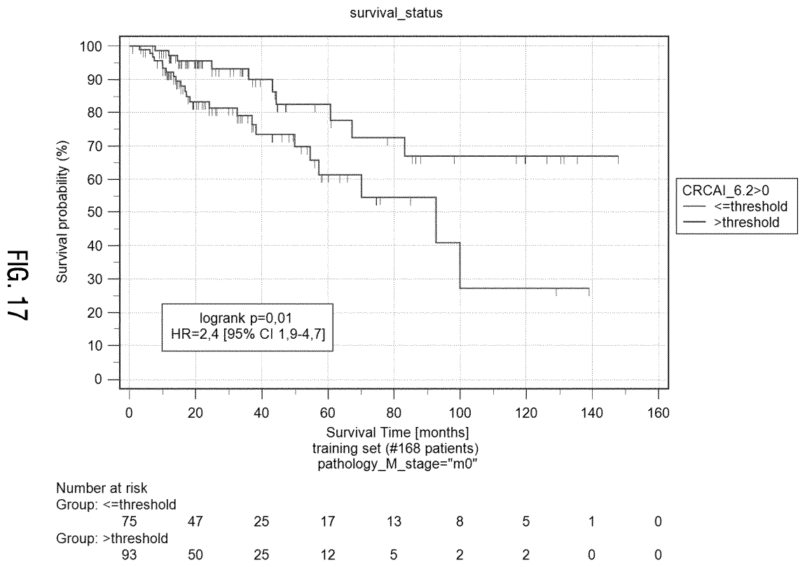

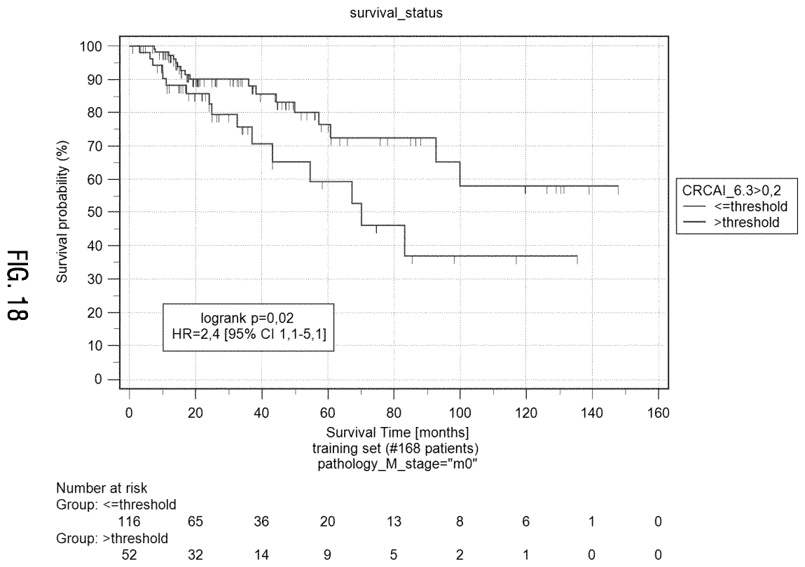

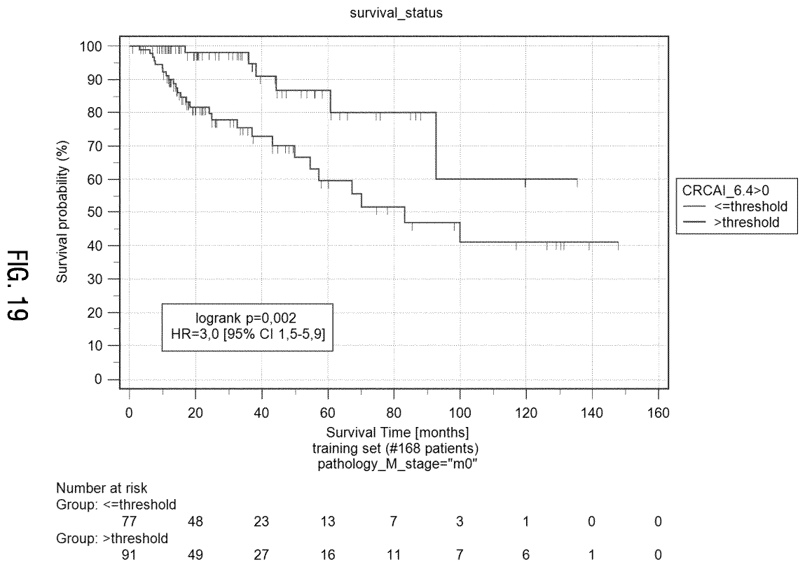

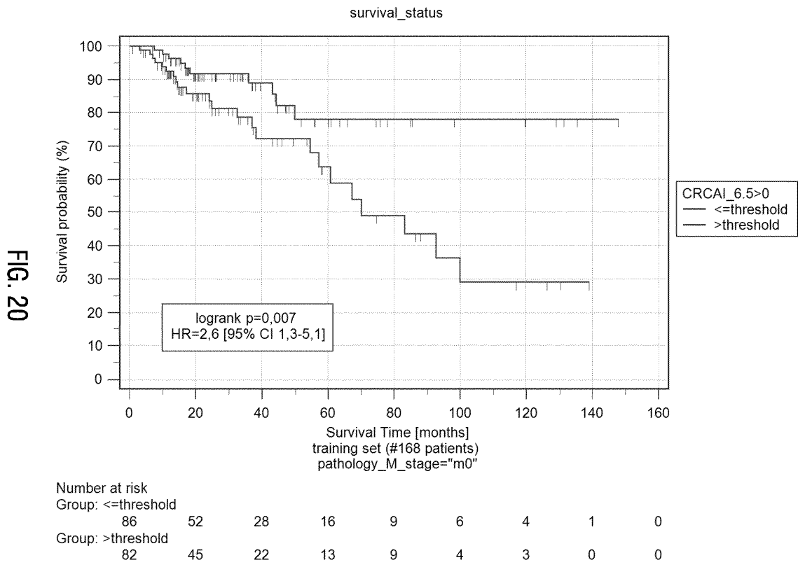

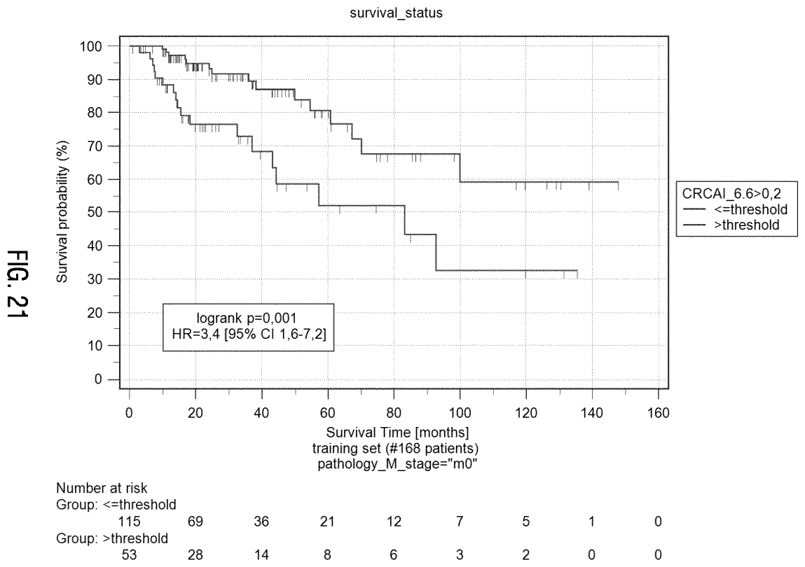

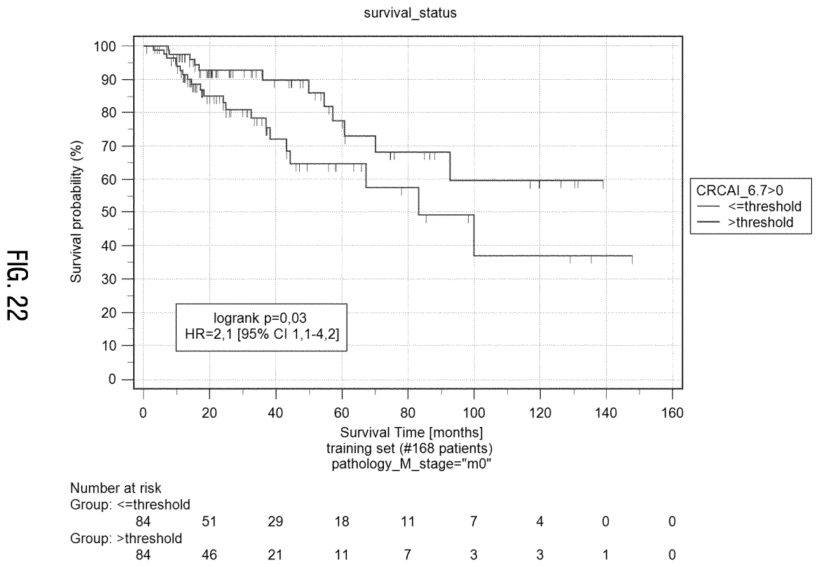

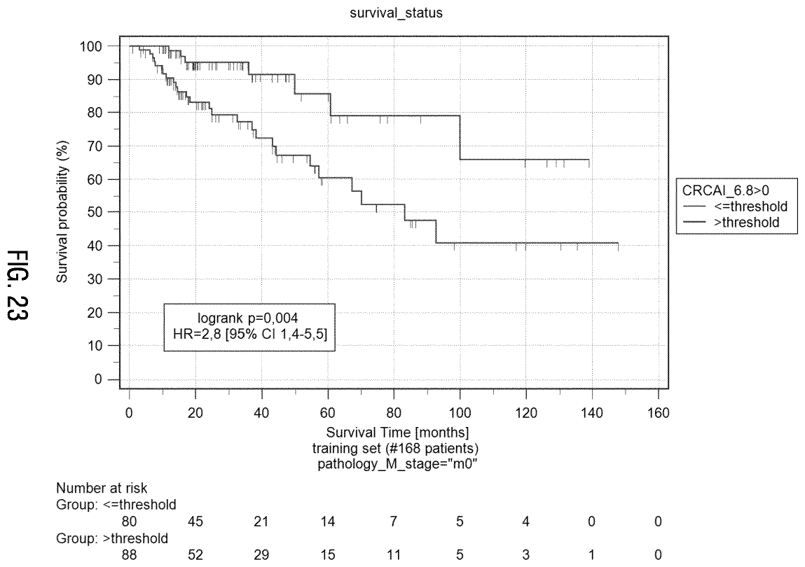

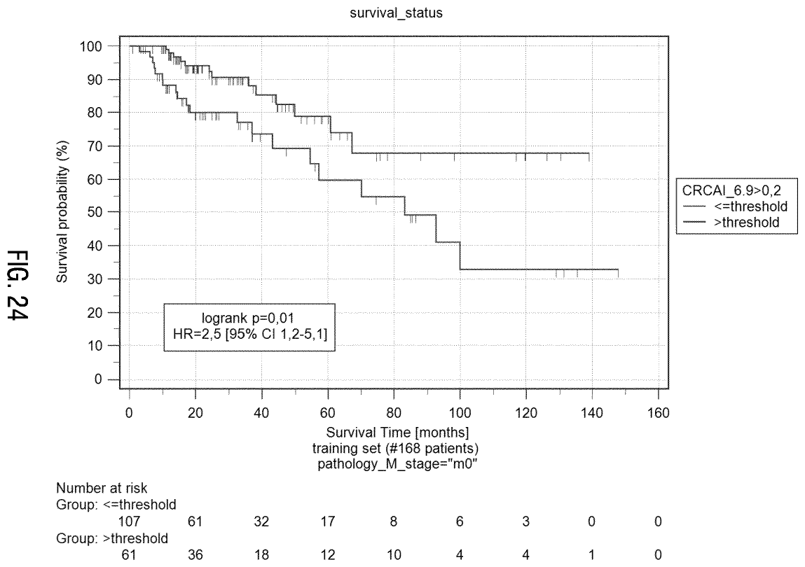

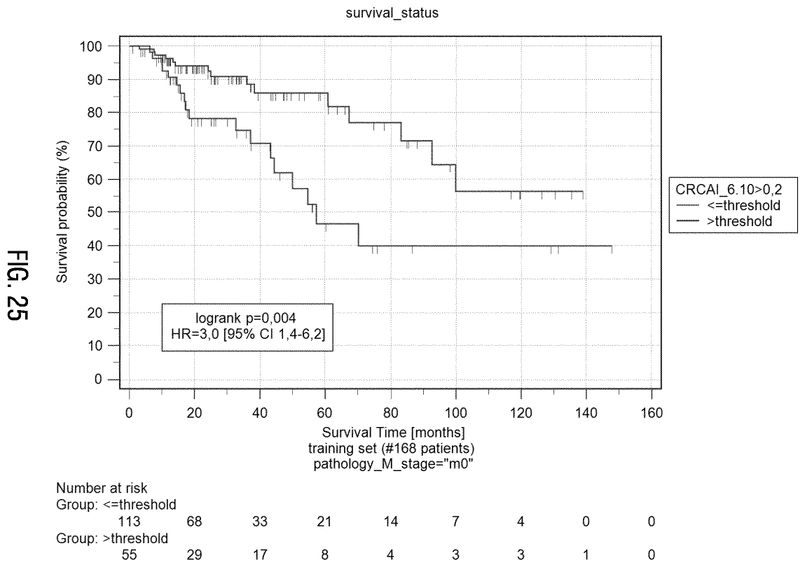

FIG. 15 shows a Kaplan-Meier curve of the CRCAI & Clinical_model in a colorectal cancer cohort derived from the GSE41248 data set. The model was trained on ⅔ of the samples from patients with non-metastasized (m0) and metastasized disease (ml) and then tested on the remaining ⅓ of the m0 and ml samples. This trained model was then tested only on samples from patients with non-metastasized disease (m0) in order to have the same situation as with the TCGA colorectal cancer cohort. The clinical endpoint that was tested was the cancer specific death (logrank p=0.0001; HR=16.8; 95% CI=6.0-47.1). The following supplementary lists indicate the number of patients at risk for the CRCAI & Clinical_model classes analyzed (threshold=0.5), i.e., the patients at risk at any time interval+20 months are shown: Low risk: 84, 76, 71, 61, 48, 31, 17, 8, 3, 2, 2, 0; High risk: 33, 29, 25, 21, 10, 7, 4, 0, 0, 0, 0, 0.

The Kaplan-Meier survival curve analysis as shown in FIGS. 2 to 15 demonstrates the presence of different patient risk groups. The risk group of a patient is determined by the probability to suffer from the respective clinical endpoint (overall death) as calculated by the respective risk model as shown in the figures. Depending on the predicted risk of a patient (i.e., depending on in which risk group the patient may belong) to die from colorectal cancer different types of interventions might be indicated. In the low risk group (probability <0.5) standard of care (SOC) delivers acceptable long-term oncological control. This is definitely not the case for the patient group with a risk >0.5 to experience any of the relevant outcomes. In this patient group escalation of intervention or application of alternative treatment needs to happen. Alternative options for treatment escalation are adjuvant therapies with radiation or cytotoxic drugs or alternative therapies like immunotherapies (e.g., atezolizumab; pembrolizumab; nivolumab; avelumab; durvalumab) or other experimental therapies.

Next it was investigated whether a smaller subset of genes would still have predictive power. In order to investigate this, ten randomly selected groups of six genes were generated that meet the following criteria: at least one gene is selected from the PDE4D7 correlated gene set. The following selections were obtained:

TABLE 3
Model name: CRCAI_6.1

| gene | weigth |
|---|---|
| ABCC5 | 0.03292 |
| SLC39A11 | −0.03793 |
| IFI6 | −0.1705 |
| IFIH1 | 0.3361 |
| CD2 | −0.07875 |
| PRKACA | 0.3371 |

TABLE 4
Model name: CRCAI_6.2

| gene | weigth |
|---|---|
| PDE4D | 0.1587 |
| RAP1GAP2 | 0.1603 |
| CIAO1 | 0.4333 |
| DHX9 | −0.2751 |
| CSK | 0.05275 |
| PTPRC | 0.1176 |

TABLE 5
Model name: CRCAI_6.3

| gene | weigth |
|---|---|
| KIAA1549 | −0.3068 |
| LRRFIP1 | 0.05373 |
| OAS1 | −0.05135 |
| TLR8 | −0.07818 |
| FYN | 0.3336 |
| LCK | 0.01731 |

TABLE 6
Model name: CRCAI_6.4

| gene | weigth |
|---|---|
| CUX2 | 0.3594 |
| APOBEC3A | 0.3644 |
| IFIT1 | 0.04772 |
| CSK | 0.1018 |
| EZR | 0.1335 |
| PTPRC | −0.3603 |

TABLE 7
Model name: CRCAI_6.5

| gene | weigth |
|---|---|
| ABCC5 | −0.001284 |
| KIAA1549 | −0.2206 |
| DDX58 | −0.02687 |
| DHX9 | −0.2461 |
| CD4 | 0.06442 |
| ZAP70 | 0.1033 |

TABLE 8
Model name: CRCAI_6.6

| gene | weigth |
|---|---|
| RAP1GAP2 | 0.0278 |
| VWA2 | −0.02628 |
| MYD88 | −0.04072 |
| OAS1 | −0.02130 |
| ZBP1 | 0.1140 |
| PRKACB | −0.3716 |

TABLE 9
Model name: CRCAI_6.7

| gene | weigth |
|---|---|
| CUX2 | 0.2735 |
| SLC39A11 | −0.1832 |
| AIM2 | −0.09114 |
| IFIT3 | 0.1104 |
| CD3E | −0.004317 |
| PAG1 | −0.03536 |

TABLE 10
Model name: CRCAI_6.8

| gene | weigth |
|---|---|
| KIAA1549 | −0.3105 |
| TDRD1 | −0.1777 |
| DHX9 | −0.3048 |
| CD28 | 0.5916 |
| CD3G | −0.3600 |
| PRKACB | −0.4245 |

TABLE 11
Model name: CRCAI_6.9

| gene | weigth |
|---|---|
| RAP1GAP2 | −0.06589 |
| APOBEC3A | 0.3345 |
| IFI6 | 0.1240 |
| LRRFIP1 | 0.07909 |
| OAS1 | −0.1445 |
| PRKACB | −0.4017 |

TABLE 12
Model name: CRCAI_6.10

| gene | weigth |
|---|---|
| TDRD1 | −0.1151 |
| CIAO1 | 0.3805 |
| IFIT3 | 0.1444 |
| CSK | 0.05788 |
| FYN | 0.3248 |
| PTPRC | −0.05306 |

This section shows additional results for Cox regression models based on a multitude of gene models for both colorectal cancer, comprising randomly selected combinations of six genes as described above in Tables 3-12. Variables and corresponding weights are provided above. The Cox regression models are plotted in the Kaplan-Meier curve analyses of FIGS. 16-25.

For Kaplan-Meier curve analysis the Cox regression function of the 10 risk models as shown above (CRCAI6.1-CRCAI6.10) were categorized into two sub-cohorts (low risk vs. high risk) based on a cut-off. The threshold for group separation into low and high risk was based on the risk to experience the clinical endpoint (outcome) as predicted by the respective Cox regression model.

FIG. 16 shows a Kaplan-Meier curve of the CRCAI_6.1_model in a 168 patient TCGA colorectal cancer cohort (training set used to develop the CRCAI_6.1_model). The clinical endpoint that was tested was the overall death (logrank p=0.03; HR=2.2; 95% CI=1.1-4.5). The supplementary lists below the graph indicate the number of patients at risk for the CRCAI_6.1 model classes analyzed (threshold=0), i.e., the patients at risk at any time interval+20 months are shown. The top line in the graph depicts patient below or equal to the threshold and the lower line in the graph depicts patients above the threshold.

FIG. 17 shows a Kaplan-Meier curve of the CRCAI_6.2_model in a 168 patient TCGA colorectal cancer cohort (training set used to develop the CRCAI_6.2_model). The clinical endpoint that was tested was the overall death (logrank p=0.01; HR=2.4; 95% CI=1.9-4.7). The supplementary lists below the graph indicate the number of patients at risk for the CRCAI_6.2 model classes analyzed (threshold=0), i.e., the patients at risk at any time interval+20 months are shown. The top line in the graph depicts patient below or equal to the threshold and the lower line in the graph depicts patients above the threshold.

FIG. 18 shows a Kaplan-Meier curve of the CRCAI_6.3_model in a 168 patient TCGA colorectal cancer cohort (training set used to develop the CRCAI_6.3_model). The clinical endpoint that was tested was the overall death (logrank p=0.02; HR=2.4; 95% CI=1.1-5.1). The supplementary lists below the graph indicate the number of patients at risk for the CRCAI_6.3 model classes analyzed (threshold=0), i.e., the patients at risk at any time interval+20 months are shown. The top line in the graph depicts patient below or equal to the threshold and the lower line in the graph depicts patients above the threshold.

FIG. 19 shows a Kaplan-Meier curve of the CRCAI_6.4_model in a 168 patient TCGA colorectal cancer cohort (training set used to develop the CRCAI_6.4_model). The clinical endpoint that was tested was the overall death (logrank p=0.002; HR=3.0; 95% CI=1.5-5.9). The supplementary lists below the graph indicate the number of patients at risk for the CRCAI_6.4 model classes analyzed (threshold=0), i.e., the patients at risk at any time interval+20 months are shown. The top line in the graph depicts patient below or equal to the threshold and the lower line in the graph depicts patients above the threshold.

FIG. 20 shows a Kaplan-Meier curve of the CRCAI_6.5_model in a 168 patient TCGA colorectal cancer cohort (training set used to develop the CRCAI_6.5_model). The clinical endpoint that was tested was the overall death (logrank p=0.007; HR=2.6; 95% CI=1.3-5.1). The supplementary lists below the graph indicate the number of patients at risk for the CRCAI_6.5 model classes analyzed (threshold=0), i.e., the patients at risk at any time interval+20 months are shown. The top line in the graph depicts patient below or equal to the threshold and the lower line in the graph depicts patients above the threshold.

FIG. 21 shows a Kaplan-Meier curve of the CRCAI_6.6_model in a 168 patient TCGA colorectal cancer cohort (training set used to develop the CRCAI_6.6_model). The clinical endpoint that was tested was the overall death (logrank p=0.001; HR=3.4; 95% CI=1.6-7.2). The supplementary lists below the graph indicate the number of patients at risk for the CRCAI_6.6 model classes analyzed (threshold=0), i.e., the patients at risk at any time interval+20 months are shown. The top line in the graph depicts patient below or equal to the threshold and the lower line in the graph depicts patients above the threshold.

FIG. 22 shows a Kaplan-Meier curve of the CRCAI_6.7_model in a 168 patient TCGA colorectal cancer cohort (training set used to develop the CRCAI_6.7_model). The clinical endpoint that was tested was the overall death (logrank p=0.03; HR=2.1; 95% CI=1.1-4.2). The supplementary lists below the graph indicate the number of patients at risk for the CRCAI_6.7 model classes analyzed (threshold=0), i.e., the patients at risk at any time interval+20 months are shown. The top line in the graph depicts patient below or equal to the threshold and the lower line in the graph depicts patients above the threshold.

FIG. 23 shows a Kaplan-Meier curve of the CRCAI_6.8_model in a 168 patient TCGA colorectal cancer cohort (training set used to develop the CRCAI_6.8_model). The clinical endpoint that was tested was the overall death (logrank p=0.004; HR=2.8; 95% CI=1.4-5.5). The supplementary lists below the graph indicate the number of patients at risk for the CRCAI_6.8 model classes analyzed (threshold=0), i.e., the patients at risk at any time interval+20 months are shown. The top line in the graph depicts patient below or equal to the threshold and the lower line in the graph depicts patients above the threshold.

FIG. 24 shows a Kaplan-Meier curve of the CRCAI_6.9_model in a 168 patient TCGA colorectal cancer cohort (training set used to develop the CRCAI_6.9_model). The clinical endpoint that was tested was the overall death (logrank p=0.01; HR=2.5; 95% CI=1.2-5.1). The supplementary lists below the graph indicate the number of patients at risk for the CRCAI_6.9 model classes analyzed (threshold=0), i.e., the patients at risk at any time interval+20 months are shown. The top line in the graph depicts patient below or equal to the threshold and the lower line in the graph depicts patients above the threshold.

FIG. 25 shows a Kaplan-Meier curve of the CRCAI_6.10_model in a 168 patient TCGA colorectal cancer cohort (training set used to develop the CRCAI_6.10_model). The clinical endpoint that was tested was the overall death (logrank p=0.004; HR=3.0; 95% CI=1.4-6.2). The supplementary lists below the graph indicate the number of patients at risk for the CRCAI_6.10 model classes analyzed (threshold=0), i.e., the patients at risk at any time interval+20 months are shown. The top line in the graph depicts patient below or equal to the threshold and the lower line in the graph depicts patients above the threshold.

From these data can be concluded that a model of six genes selected from the Immune defense response signature, the T-cell signaling signature and the PDE4D7 correlated signature as described herein, considering at least gene from the six genes is selected from the PDE4D7 correlated gene signature, suffices to make a prediction. Because 10 randomly selected sets of six genes meeting these criteria resulted in a significant risk stratification of patients, it is anticipated that these results can be extrapolated to any selection of six of more genes.

### DISCUSSION

The effectiveness of therapies for colorectal cancers is limited, resulting in disease progression and ultimately death of patients, especially for those at high risk of recurrence of disease after primary intervention. The prediction of the therapy outcome is very challenging as many factors play a role in therapy effectiveness and disease recurrence. It is likely that important factors have not yet been identified, while the effect of others cannot be determined precisely. Multiple clinico-pathological measures are currently investigated and applied in a clinical setting to improve response prediction and therapy selection, providing some degree of improvement. Nevertheless, a strong need remains for better prediction of the treatment response in order to increase the success rate of colorectal cancer therapies.

We have identified molecules of which expression shows a significant relation to mortality after primer therapy of colorectal cancers and therefore are expected to improve the prediction of the effectiveness of secondary treatments. This can be achieved by 1) standard of care for those patients with low risk of progressive disease correlated with death from cancer and/or 2) guiding patients with high risk of progressive disease and subsequent death from cancer to an alternative, potentially more effective form of treatment as compared to currently applied standard of care. This would reduce suffering for those patients who would be spared ineffective therapy and would reduce cost spent on ineffective therapies.

Other variations to the disclosed realizations can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

One or more steps of the method illustrated in FIG. 1 may be implemented in a computer program product that may be executed on a computer. The computer program product may comprise a non-transitory computer-readable recording medium on which a control program is recorded (stored), such as a disk, hard drive, or the like. Common forms of non-transitory computer-readable media include, for example, floppy disks, flexible disks, hard disks, magnetic tape, or any other magnetic storage medium, CD-ROM, DVD, or any other optical medium, a RAM, a PROM, an EPROM, a FLASH-EPROM, or other memory chip or cartridge, or any other non-transitory medium from which a computer can read and use.

Alternatively, the one or more steps of the method may be implemented in transitory media, such as a transmittable carrier wave in which the control program is embodied as a data signal using transmission media, such as acoustic or light waves, such as those generated during radio wave and infrared data communications, and the like.

The exemplary method may be implemented on one or more general purpose computers, special purpose computer(s), a programmed microprocessor or microcontroller and peripheral integrated circuit elements, an ASIC or other integrated circuit, a digital signal processor, a hardwired electronic or logic circuit such as a discrete element circuit, a programmable logic device such as a PLD, PLA, FPGA, Graphical card CPU (GPU), or PAL, or the like. In general, any device, capable of implementing a finite state machine that is in turn capable of implementing the flowchart shown in FIG. 1, can be used to implement one or more steps of the method of risk stratification for therapy selection in a patient with prostate cancer is illustrated. As will be appreciated, while the steps of the method may all be computer implemented, in some embodiments one or more of the steps may be at least partially performed manually.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified herein.

Any reference signs in the claims should not be construed as limiting the scope.

The invention relates to a method of predicting an outcome of a colorectal cancer subject, comprising:
determining or receiving the result of a determination of six or more gene expression levels selected from a first gene expression profile, a second gene expression profile and a third gene expression profile, wherein said six or more gene expression levels comprise at least one or more gene expression level selected from the third gene expression profile and at least one or more gene expression level selected from the first and/or the second gene expression profile, wherein
the first gene expression profile consist of the immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1;
the second gene expression profile consist of the T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70;
the third gene expression profile consists of the PDE4D7 correlated genes selected from the group consisting of: ABCC5, CUX2, KIAA1549, PDE4D, RAP1GAP2, SLC39A11, TDRD1, and VWA2;
said first, second and third gene expression profiles being determined in a biological sample obtained from the subject; and
determining the prediction of the outcome based on six or more gene expression levels, and
optionally, providing the prediction to a medical caregiver or the subject. In an embodiment the six or more gene expression levels comprise one or more, for example, 1, 2, 3, 4, 5, 6, 7, 8, 9, 10, 11, 12, 13 or all, immune defense response genes, and one or more, for example, 1, 2, 3, 4, 5, 6, 7, 8, 9, 10, 11, 12, 13 or all, T-Cell receptor signaling genes, and one or more, for example, 1, 2, 3, 4, 5, 6, 7, 8, 9, 10, 11, 12, 13 or all, PDE4D7 correlated genes.

Alternatively, the invention relates to a method of predicting an outcome of a colorectal cancer subject is presented, comprising determining or receiving the result of a determination of a first gene expression profile for each of one or more, for example, 1, 2, 3, 4, 5, 6, 7, 8, 9, 10, 11, 12, 13 or all, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, said first gene expression profile(s) being determined in a biological sample obtained from the subject, and/or determining or receiving the result of a determination of a second gene expression profile for each of one or more, for example, 1, 2, 3, 4, 5, 6, 7, 8, 9, 10, 11, 12, 13, 14, 15, 16 or all, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, said second gene expression profile(s) being determined in a biological sample obtained from the subject, and/or determining or receiving the result of a determination of a third gene expression profile for each of one or more, for example, 1, 2, 3, 4, 5, 6, 7 or all, PDE4D7 correlated genes selected from the group consisting of: ABCC5, CUX2, KIAA1549, PDE4D, RAP1GAP2, SLC39A11, TDRD1, and VWA2, said third gene expression profile(s) being determined in a biological sample obtained from the subject, determining the prediction of outcome based on the first gene expression profile(s), or on the second gene expression profile(s), or on the third gene expression profile(s), or on the first, second, and third gene expression profile(s), and, optionally, providing the prediction to a medical caregiver or the subject.

In some embodiments, the prediction of the outcome can also be determined based on the first gene expression profile(s) and the second genes expression profile(s). In some embodiments, the prediction of the outcome can also be determined based on the first gene expression profile(s) and the third genes expression profile(s). In some embodiments, the prediction of the outcome can also be determined based on the second gene expression profile(s) and the third genes expression profile(s).

The attached Sequence Listing, entitled 2020PF00762_Sequence Listing_ST25 is incorporated herein by reference, in its entirety.

1. A method of predicting an outcome of a colorectal cancer subject, comprising:
determining or receiving the result of a determination of six or more gene expression levels selected from a first gene expression profile, a second gene expression profile and a third gene expression profile, wherein said six or more gene expression levels comprise at least one or more gene expression level selected from the third gene expression profile and at least one or more gene expression level selected from the first and/or the second gene expression profile, wherein
the first gene expression profile consist of the immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1;
the second gene expression profile consist of the T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70;
the third gene expression profile consists of the PDE4D7 correlated genes selected from the group consisting of: ABCC5, CUX2, KIAA1549, PDE4D, RAP1GAP2, SLC39A11, TDRD1, and VWA2;
said first, second and third gene expression profiles being determined in a biological sample obtained from the subject; and
determining the prediction of the outcome based on six or more gene expression levels.

2. The method as defined in claim 1, wherein:
the six or more gene expression levels comprise:
one or more immune defense response genes, and
one or more T-Cell receptor signaling genes, and
one or more PDE4D7 correlated genes.

3. The method as defined in claim 1, wherein the determining of the prediction of the outcome comprises:
combining the first gene expression profiles for two or more of the immune defense response genes with a regression function that had been derived from a population of colorectal cancer subjects, and/or
combining the second gene expression profiles for two or more of the T-Cell receptor signaling genes with a regression function that had been derived from a population of colorectal cancer subjects, and/or
combining the third gene expression profiles for two or more of the PDE4D7 correlated genes with a regression function that had been derived from a population of colorectal cancer subjects, and/or
combining the six or more gene expression levels or all gene expression levels with a regression function that had been derived from a population of colorectal cancer subjects.

4. The method as defined in claim 3, wherein the determining of the prediction of the outcome further comprises combining the combination of the first gene expression profiles, the combination of the second gene expression profiles, and the combination of the third gene expression profiles with a regression function that had been derived from a population of colorectal cancer subjects.

5. The method as defined in claim 1, wherein the determining of the outcome is further based on one or more clinical parameters obtained from the subject.

6. The method as defined in claim 4, wherein the clinical parameters comprise one or more of: (i) T stage attribute (T1, T2, T3, or T4); (ii) N stage attribute (NO, N1, or N2), and; (iii) M stage attribute (M0, M1).

7. The method as defined in claim 5, wherein the determining of the prediction of the outcome comprises combining one or more of: (i) the first gene expression profile(s) for the one or more immune defense response genes; (ii) the second gene expression profile(s) for the one or more T-Cell receptor signaling genes; (iii) the third gene expression profile(s) for the one or more PDE4D7 correlated genes, and; (iv) the combination of the first gene expression profiles, the combination of the second gene expression profiles, and the combination of the third gene expression profiles, and the one or more clinical parameters obtained from the subject with a regression function that had been derived from a population of colorectal cancer subjects.

8. The method as defined in claim 1, wherein the biological sample is obtained from the subject before the start of the therapy.

9. The method as defined in claim 1, wherein the therapy is surgery, radiotherapy, cytotoxic chemotherapy (CTX), short- or long-course chemo-radiation therapy (CRT), immunotherapy, or any combination thereof.

10. The method as defined in claim 1, wherein the prediction of the therapy response is unlikely or likely for the effectiveness of the therapy, wherein a therapy is recommended based on the prediction and, if the prediction is unlikely, the recommended therapy comprises one or more of: (i) therapy provided earlier than is the standard; (ii) radiotherapy with an increased effective dose; (iii) an adjuvant therapy; (iv) long-course CRT (chemo-radiation therapy), and (iv); an alternative therapy.

11. An apparatus for predicting an outcome of a colorectal cancer subject, comprising:
an input adapted to receive data indicative of a first gene expression profile for each of one or more, for example, immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, said first gene expression profile(s) being determined in a biological sample obtained from the subject, and/or of a second gene expression profile for each of one or more, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, said second gene expression profile(s) being determined in a biological sample obtained from the subject, and/or of a third gene expression profile for each of one or more, PDE4D7 correlated genes selected from the group consisting of: ABCC5, CUX2, KIAA1549, PDE4D, RAP1GAP2, SLC39A11, TDRD1, and VWA2, said third gene expression profile(s) being determined in a biological sample obtained from the subject, and
a processor adapted to determine the prediction of outcome based on the first gene expression profile(s), or on the second gene expression profile(s), or on the third gene expression profile(s), or on the first, second, and third gene expression profile(s).

12. A non-transitory computer program product comprising instructions which, when the program is executed by a computer, cause the computer to carry out a method comprising:
receiving data indicative of a first gene expression profile for each of one or more, for example immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, said first gene expression profile(s) being determined in a biological sample obtained from a colorectal cancer subject, and/or of a second gene expression profile for each of one or more, T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70, said second gene expression profile(s) being determined in a biological sample obtained from a colorectal cancer subject, and/or of a third gene expression profile for each of one or more, PDE4D7 correlated genes selected from the group consisting of: ABCC5, CUX2, KIAA1549, PDE4D, RAP1GAP2, SLC39A11, TDRD1, and VWA2, said third gene expression profile(s) being determined in a biological sample obtained from a colorectal cancer subject, and
determining a prediction of an outcome of the subject based on the first gene expression profile(s), or on the second gene expression profile(s), or on the third gene expression profile(s), or on the first, second, and third gene expression profile(s).

13. A diagnostic kit, comprising:
at least one primer and/or probe for determining in a biological sample obtained from a subject, six or more gene expression levels selected from a first gene expression profile, a second gene expression profile and a third gene expression profile, wherein said six or more gene expression levels comprise at least one or more gene expression level selected from the third gene expression profile and at least one or more gene expression level selected from the first and/or the second gene expression profile, wherein
the first gene expression profile consists of the immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1;
the second gene expression profile consists of the T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70; and
the third gene expression profile consisting of the PDE4D7 correlated genes selected from the group consisting of: ABCC5, CUX2, KIAA1549, PDE4D, RAP1GAP2, SLC39A11, TDRD1, and VWA2.

14. A method of using the kit as defined in claim 13 for predicting an outcome of a colorectal cancer subject.

15. The method as defined in claim 14 wherein predicting an outcome of a colorectal cancer subject comprises determining or receiving the result of a determination of six or more gene expression levels selected from a first gene expression profile, a second gene expression profile and a third gene expression profile.

16. A method, comprising:
receiving one or more biological sample(s) obtained from a colorectal cancer subject,
using the kit as defined in claim 13 to determine six or more gene expression levels selected from a first gene expression profile, a second gene expression profile and a third gene expression profile, wherein said six or more gene expression levels comprise at least one or more gene expression level selected from the third gene expression profile and at least one or more gene expression level selected from the first and/or the second gene expression profile, wherein
the first gene expression profile consists of the immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1;
the second gene expression profile consists of the T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70; and
the third gene expression profile consists of the PDE4D7 correlated genes selected from the group consisting of: ABCC5, CUX2, KIAA1549, PDE4D, RAP1GAP2, SLC39A11, TDRD1, and VWA2.

17. A method if using of six or more gene expression levels selected from a first gene expression profile, a second gene expression profile and a third gene expression profile, wherein said six or more gene expression levels comprise at least one or more gene expression level selected from the third gene expression profile and at least one or more gene expression level selected from the first and/or the second gene expression profile, wherein the first gene expression profile consists of the immune defense response genes selected from the group consisting of: AIM2, APOBEC3A, CIAO1, DDX58, DHX9, IFI16, IFIH1, IFIT1, IFIT3, LRRFIP1, MYD88, OAS1, TLR8, and ZBP1, the second gene expression profile consists of the T-Cell receptor signaling genes selected from the group consisting of: CD2, CD247, CD28, CD3E, CD3G, CD4, CSK, EZR, FYN, LAT, LCK, PAG1, PDE4D, PRKACA, PRKACB, PTPRC, and ZAP70 the third gene expression profile consists of the PDE4D7 correlated genes selected from the group consisting of: ABCC5, CUX2, KIAA1549, PDE4D, RAP1GAP2, SLC39A11, TDRD1, and VWA2, in a method of predicting an outcome of a colorectal cancer subject, comprising:
determining the prediction of the outcome based on the six or more gene expression levels, and
providing the prediction to a medical caregiver or the subject.

18. The method of claim 1, further comprising transmitting the prediction to a device associate with one or more of: a medical caregiver or the subject

19. The method of claim 2, wherein the one or more immune defense response genes is two or more immune defense genes, and the one or more T-Cell receptor signaling genes is two or more T-Cell receptor signaling genes, and the one or more PDE4D7 correlated genes is two or more PDE4D7 correlated genes.

20. The method of claim 10, wherein an adjuvant therapy is chemotherapy and wherein an alternative therapy is immunotherapy.
